(19) United States
(12) Patent Application Publication
(10) Pub. No.: US 2018/0057601 A1
(43) Pub. Date: Mar. 1, 2018

(54) NOVEL HUMANIZED ADAM17 ANTIBODY

(71) Applicant: PIERRE FABRE MEDICAMENT, Boulogne Billancourt (FR)

(72) Inventors: Peter LOWE, Chazey Bons (FR); Sven BERGER, Poisy (FR); Michael TESAR, Friedberg (DE)

(21) Appl. No.: 15/538,331

(22) PCT Filed: Jan. 4, 2016
(86) PCT No.: PCT/EP2016/050029

§ 371 Date: Jun. 21, 2017

(30) Foreign Application Priority Data
Dec. 24, 2014 (EP) 14307185.0

(51) Int. Cl. C07K 16/28; C07K 16/30

(52) U.S. Cl. CPC C07K 16/2896; C07K 16/30; C07K 2317/24; C07K 2317/56; A61K 2039/505; C07K 2317/567; C07K 2317/55; C07K 2317/54; C07K 2317/622; C07K 2317/565

(57) ABSTRACT

The present disclosure relates to a novel humanized antibody capable of binding to ADAM17 and also the amino and nucleic acid sequences coding for said antibody. From one aspect, the disclosure relates to a novel humanized antibody, or antigen-binding fragments, capable of binding to ADAM17 with anti-tumour activities. The disclosure also comprises the use of said humanized antibody as a drug for the treatment of cancer. Finally, the invention comprises compositions comprising said humanized antibody, alone or in combination or conjugation with other anticancer compounds, and the use of same for the treatment of cancer.

The present invention relates to a humanized antibody capable of binding to ADAM17 and also the amino and nucleic acid sequences coding for said humanized antibody. The invention also comprises the use of said humanized antibody as a medicament for the treatment of cancer. The invention encompasses compositions comprising said humanized antibody, alone or in combination or conjugation with other anti-cancer compounds, and the use of same for the treatment of cancer.

ADAM17 (A disintegrin and metalloproteinase domain-containing protein 17) also referred to as Snake venom-like protease, TNF-alpha convertase, TNF-alpha-converting enzyme (TACE) and CD156b is a membrane bound metalloprotease responsible for the extracellular cleavage (ectodomain shedding) of a number of pathologically important substrates. Originally identified as the enzyme responsible for the cleavage of membrane bound pro-TNF-α liberating soluble protein, ADAM17 has since been described in the ectodomain shedding of a large number of membrane bound precursors protein. Ectodomain shedding by ADAM17 releases from the membrane of cells a large number of soluble cytokines and growth factors such as: Amphiregulin, Heparin binding-EGF like growth factor (HB-EGF), Transforming growth factor alpha (TGF-α), epiregulin, epigen and neuregulins. ADAM17 also mediates the shedding of numerous receptors including; IL-6Rα, IL-1RII, Her4, c-Kit, Notch, Mer, TNF-α RI & II where the physiological result can be signal silencing through receptor shedding or soluble ligand trapping, or receptor transactivation as is described for IL-6Rα and gp130. ADAM17 can actively participate in the remodelling of the extracellular matrix and cell-cell contacts through the shedding of a large number of adhesion molecules and constituents of the extracellular microenvironment such as: L-selectin, ICAM-1, VCAM-1, Nectin-4, CD44 and collagen XVII. Less well understood activities of ADAM17 include the ectodomain shedding of cellular prion protein and amyloid precursor protein.

For the avoidance of doubt, without any specification, the expression ADAM17 refers to the human ADAM17 of sequence SEQ ID No. 22.

Structurally, ADAM17 consists of an 824 amino acids protein comprising a preproprotein domain (aa 1-214), an extracellular domain (aa 215-671), a transmembrane domain (aa 672-692) and a cytoplasmic domain (aa 693-824).

More particularly, the extracellular domain is comprised of a Metalloprotease (MP) domain of sequence SEQ ID No. 23 (corresponding to aa 215-474 of ADAM17), a Disintegrin (DI) domain of sequence SEQ ID No. 24 (corresponding to aa 475-563 of ADAM17) and a Membrane proximal (MPD) domain of sequence SEQ ID No. 25 (corresponding to aa 564-671 of ADAM17).

ADAM17 has been described as a technically difficult target for the generation of antagonistic antibodies indeed complex selection strategies have been described that necessitate selection and optimisation of specific binders to generate the desired characteristics. To date only one such specific antagonist has been described: the antibody D1(A12) capable of binding to ADAM17 through the disintegrin-cysteine rich and catalytic domains simultaneously. The antagonistic activity of D1(A12) is dependent on binding both the disintegrin-cysteine rich and catalytic domain to demonstrate its antagonistic activity.

The present invention intends to remedy the lack of relevant antibodies targeting ADAM17 with an efficient anti-tumoural activity.

In a first aspect, the invention relates to a humanized antibody, or an antigen-binding fragment thereof, that binds to ADAM17.

Humanisation of mouse monoclonal antibodies was initially achieved by grafting the complementarity-determining regions (CDRs) of mouse monoclonal antibodies onto human framework regions of the variable antibody domains of human antibodies. In addition, several amino acid residues present in those framework regions were identified as interacting with the CDRs or antigen and may be back mutated in the humanized antibody to improve binding or any other property.

The humanized antibody, or any antigen-binding fragment thereof, can also be described as comprising: i) a heavy chain comprising CDR-H1, CDR-H2 and CDR-H3 comprising respectively amino acid sequences SEQ ID Nos. 1, 2 and 3, or sequences with at least 80%, preferably 85%, 90%, 95% and 98% identity after optimal alignment with sequences SEQ ID Nos. 1, 2 and 3; and ii) a light chain comprising CDR-L1, CDR-L2 and CDR-L3 comprising respectively amino acid sequences SEQ ID Nos. 4, 5 and 6, or sequences with at least 80%, preferably 85%, 90%, 95% and 98% identity after optimal alignment with sequences SEQ ID Nos. 4, 5 and 6.

For the avoidance of doubt, without any contrary indication in the text, the expression CDRs means the hypervariable regions of the heavy and light chains of an antibody as defined by IMGT.

The IMGT unique numbering has been defined to compare the variable domains whatever the antigen receptor, the chain type, or the species [Lefranc M.-P., Immunology Today 18, 509 (1997)/Lefranc M.-P., The Immunologist, 7, 132-136 (1999)/Lefranc, M.-P., Pommié, C., Ruiz, M., Giudicelli, V., Foulquier, E., Truong, L., Thouvenin-Contet, V. and Lefranc, Dev. Comp. Immunol., 27, 55-77 (2003)]. In the IMGT unique numbering, the conserved amino acids always have the same position, for instance cystein 23 (1st-CYS), tryptophan 41 (CONSERVED-TRP), hydrophobic amino acid 89, cystein 104 (2nd-CYS), phenylalanine or tryptophan 118 (J-PHE or J-TRP). The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. As gaps represent unoccupied positions, the CDR-IMGT lengths (shown between brackets and separated by dots, e.g. [8.8.13]) become crucial information. The IMGT unique numbering is used in 2D graphical representations, designated as IMGT Colliers de Perles, and in 3D structures in IMGT/3Dstructure-DB.

Three heavy chain CDRs and 3 light chain CDRs exist. The term CDR or CDRs is used here in order to indicate, according to the case, one of these regions or several, or even the whole, of these regions which contain the majority of the amino acid residues responsible for the binding by affinity of the antibody for the antigen or the epitope which it recognizes.

In the sense of the present invention, the “percentage identity” or “% identity” between two sequences of nucleic acids or amino acids means the percentage of identical nucleotides or amino acid residues between the two sequences to be compared, obtained after optimal alignment, this percentage being purely statistical and the differences between the two sequences being distributed randomly along their length. The comparison of two nucleic acid or amino acid sequences is traditionally carried out by comparing the sequences after having optimally aligned them, said comparison being able to be conducted by segment or by using an “alignment window”. Optimal alignment of the sequences for comparison can be carried out, in addition to comparison by hand, by means of the local homology algorithm of Smith and Waterman (1981), by means of the similarity search method of Pearson and Lipman (1988) or by means of computer software using these algorithms (GAP, BESTFIT, FASTA and TFASTA in the Wisconsin Genetics Software Package, Genetics Computer Group, 575 Science Dr., Madison, Wis., or by the comparison software BLAST NR or BLAST P).

The percentage identity between two nucleic acid or amino acid sequences is determined by comparing the two optimally-aligned sequences in which the nucleic acid or amino acid sequence to compare can have additions or deletions compared to the reference sequence for optimal alignment between the two sequences. Percentage identity is calculated by determining the number of positions at which the amino acid, nucleotide or residue is identical between the two sequences, preferably between the two complete sequences, dividing the number of identical positions by the total number of positions in the alignment window and multiplying the result by 100 to obtain the percentage identity between the two sequences.

For example, the BLAST program, “BLAST 2 sequences” (Tatusova et al., “Blast 2 sequences—a new tool for comparing protein and nucleotide sequences”, FEMS Microbiol., 1999, Lett. 174:247-250) available on the site http://www.ncbi.nlm.nih.gov/gorf/bl2.html, can be used with the default parameters (notably for the parameters “open gap penalty”: 5, and “extension gap penalty”: 2; the selected matrix being for example the “BLOSUM 62” matrix proposed by the program); the percentage identity between the two sequences to compare is calculated directly by the program.

For the amino acid sequence exhibiting at least 80%, preferably 85%, 90%, 95% and 98% identity with a reference amino acid sequence, preferred examples include those containing the reference sequence, certain modifications, notably a deletion, addition or substitution of at least one amino acid, truncation or extension. In the case of substitution of one or more consecutive or non-consecutive amino acids, substitutions are preferred in which the substituted amino acids are replaced by “equivalent” amino acids. Here, the expression “equivalent amino acids” is meant to indicate any amino acids likely to be substituted for one of the structural amino acids without however modifying the biological activities of the corresponding antibodies and of those specific examples defined below.

Equivalent amino acids can be determined either on their structural homology with the amino acids for which they are substituted or on the results of comparative tests of biological activity between the various antibodies likely to be generated.

As a non-limiting example, table 1 below summarizes the possible substitutions likely to be carried out without resulting in a significant modification of the biological activity of the corresponding modified antibody; inverse substitutions are naturally possible under the same conditions.

TABLE 1

| Original | |
|---|---|
| residue | Substitution(s) |
| Ala (A) | Val, Gly, Pro, Ser, Thr |
| Arg (R) | Lys, His, Gln |
| Asn (N) | Gln, Asp, His, Lys, Ser, Thr |
| Asp (D) | Glu, Asn |
| Cys (C) | Ser |
| Gln (Q) | Asn, Arg, Glu, His, Lys, Met |
| Glu (G) | Asp, Gln, Lys |
| Gly (G) | Ala, Pro |
| His (H) | Arg, Asn, Gln, Tyr |
| Ile (I) | Leu, Val, Met |
| Leu (L) | Ile, Val, Met, Phe |
| Lys (K) | Arg, Gln, Glu, Asn |
| Met (M) | Leu, Ile, , Gln, Val |
| Phe (F) | Tyr, Met, Leu, Trp |
| Pro (P) | Ala |
| Ser (S) | Thr, Cys, Ala, Asn |
| Thr (T) | Ser, Ala, Asn |
| Trp (W) | Tyr, Phe |
| Tyr (Y) | Phe, Trp, His |
| Val (V) | Leu, Ala, Ile, Met |

An embodiment of the invention relates to a humanized antibody that binds to an ADAM 17 epitope, or an antigen binding fragment thereof, characterized in that it comprises:

a) a heavy chain variable domain having i) complementary determining regions CDR-H1, CDR-H2 and CDR-H3 of sequences SEQ ID Nos. 1, 2 and 3, respectively, and ii) framework regions FR1, FR2 and FR3 derived from the human germline IGHV4-4*07 (SEQ ID No. 18), and iii) framework region FR4 derived from the human germline IGHJ6*01 (SEQ ID No. 20); and

b) a light chain variable domain having i) complementary determining regions CDR-L1, CDR-L2 and CDR-L3 of sequences SEQ ID Nos. 4, 5 and 6, respectively, and ii) framework regions FR1, FR2 and FR3 derived from the human germline IGKV1-39*01 (SEQ ID No. 19), and iii) framework region FR4 derived from the human germline IGKJ4*01 (SEQ ID No. 21).

The term “germline or “germline sequence” refers to an amino acid sequence encoded by unrearranged immunoglobulin V, D and/or J regions, or portions thereof, present in the genomic DNA of an organism.

A germline represents the basic genetic message as is transmitted from parent to sibling prior to any functional rearrangement in the coding sequence of said gene. In the case of antibodies homologous recombination of individual germline genes encoding variable (V), diversity (D, specific to heavy chains) and joining genes (J) produces a non germline gene product; VDJ for heavy chains and VJ for light chains. The potential for nucleotide insertion and deletion at the stage of germline gene recombination is responsible for the almost limitless diversity of antibodies that can be produced by the human immune repertoire. Once recombined the assembled genes form the functional immunoglobulin gene that is no longer transmisable from parent to sibling and is thus no longer a germline entity.

Germline sequences for human V, D and J genes can be obtained and interrogated from curated databases such as IMGT, UNSWIg, NCBI and VBASE2.

The source of germline sequences used in the present specification is the IMGT V, D and J germline databases. For the avoidance of doubt, the germlines IGHV4-4*07, IGHJ6*01, IGKV1-39*01 and IGKJ4*01 have been obtained from the IMGT germline database.

As used herein the term “derived from” a designated sequence refers to the origin of the sequence. In one embodiment, the amino acid sequence which is derived from a particular starting amino acid sequence has an amino acid sequence that is essentially identical to that of the starting sequence, or a portion thereof wherein the portion consists of at least of at least 3-5 amino acids, 5-10 amino acids, at least 10-20 amino acids, at least 20-30 amino acids, or at least 30-50 amino acids, or which is otherwise identifiable to one of ordinary skill in the art as having its origin in the starting sequence.

In the present specification, the expression “parental antibody” will be used to design the murine antibody from which the humanized antibody according to the invention is derived.

In an embodiment of the invention, the humanized antibody may comprise 1, 2, 3, 4, 5 or 6 of the CDRs of the heavy and/or light chain(s) of the parental antibody and the framework regions of the selected germlines. In another embodiment of the invention, the humanized antibody may comprise the 6 CDRs of the heavy and the light chain(s) of the parental antibody and the framework regions of the selected germlines.

The humanized antibody disclosed herein may also comprise one or more amino acid substitutions, insertions and/or deletions in the framework regions of the heavy and/or the light chain variable domains as compared to the corresponding germline sequences. Such mutations can be readily ascertained by comparing the amino acid sequences of the parental antibody to the germline sequences. The present invention includes antibodies, and antigen-binding fragments thereof, which are derived from any of the amino acid sequences of the germlines, wherein one or more amino acids within one or more framework regions are back-mutated to the corresponding murine residue(s) or to a conservative amino acid substitution (natural or non-natural) of the corresponding murine residue(s) (such sequence changes are, referred to herein as “back-mutations”). A person of ordinary skill in the art, starting with the heavy and light chain variable region sequences of the selected germline, can easily produce numerous antibodies and antigen-binding fragments which comprise one or more individual back-mutations or combinations thereof. In certain embodiments, all of the framework residues within the V.sub.H and/or V.sub.L domains are mutated back to the murine sequence. In other embodiments, only certain residues are mutated back to the murine sequence. Furthermore, the antibodies of the present invention may contain any combination of two or more back-mutations within the framework regions, i.e., wherein certain individual residues are mutated back to the murine sequence while certain other residues that differ from the murine sequence are maintained. Once obtained, antibodies and antigen-binding fragments that contain one or more back-mutations can be easily tested for one or more desired property such as, improved binding specificity, increased binding affinity, improved or enhanced antagonistic or agonistic biological properties (as the case may be), reduced immunogenicity, improved folding, improved expression, improved stability, improved light/heavy chain assembly, etc. Antibodies and antigen-binding fragments obtained in this general manner are encompassed within the present invention.

As non limitative example, it is hereinafter mentioned some outline of the humanisation Approach:

Step 1. Selection of either a VL human germline family or a VH human germline family with the highest homology to the parental antibody.

Step 2. Selection of either a corresponding VH human germline family or VL human germline family to create a VH/VL combination with a high frequency in the human antibody repertoire or which are know to give favourable biophysical characteristics like correct VH-VL assembly. This includes other human germline families than the one with the highest homology.

Step 3. Selection of a member of this human germline family which has the highest homology with the parental antibody in the framework regions.

Step 4. Generation of a variant in which all framework positions are derived from the selected human germlines and evaluation of its activity and biophysical properties.

Step 5. Insertion of parental residues in the frameworks in position which are identified to interact with the CDRs or have strong structural implication based a corresponding protein crystal structure model.

Step 6. Evaluation of individual and combinations of variants containing specific backmutations.

Step 7. Generation of different variants based on the results of the first evaluation which contain a different combination of backmutations and a reduced number of backmutations.

An aspect of the invention consists of a humanized antibody, or an antigen binding fragment thereof, characterized in that i) the heavy chain variable domain consists of the sequence SEQ ID No. 7 and ii) the light chain variable domain consists of the sequence SEQ ID No. 8.

As it will be clear for a person skilled in the art, sequences SEQ ID No. 7 and 8 are corresponding to consensus sequences wherein the potential back-mutations according to the invention are identified. The antibody according to the invention may comprise none or at least one back mutation(s).

In an embodiment, the heavy chain of the antibody herein described may comprise 1, 2, 3, 4, 5, 6, 7, 8, 9, 10, 11, 12, 13, 14, 15, 16, 17, 18, 19, 20, 21, 22, 23, 24, 25, 26, 27 or 28 back-mutations as identified in the consensus sequence.

In an embodiment, the light chain of the antibody herein described may comprise 1, 2, 3, 4, 5, 6, 7, 8, 9, 10, 11, 12, 13, 14, 15, 16, 17, 18, 19, 20, 21, 22, 23 or 24 back-mutations as identified in the consensus sequence.

A particular embodiment of the invention is a humanized antibody, or an antigen binding fragment thereof, characterized in that the heavy chain variable domain consists of the sequence SEQ ID No. 9, or any sequence exhibiting at least 80% identity with SEQ ID No. 9.

For the avoidance of doubt, the humanized antibody comprising the heavy chain variable domain consisting of the sequence SEQ ID No. 9 above mentioned also comprises a light chain variable domain having the CDR-L1, CDR-L2 and CDR-L3 of sequences SEQ ID Nos. 4, 5 and 6, respectively.

Another particular embodiment of the invention is a humanized antibody, or an antigen binding fragment thereof, characterized in that the light chain variable domain consists of the sequence SEQ ID No. 10, or any sequence exhibiting at least 80% identity with SEQ ID No. 10.

For the avoidance of doubt, the humanized antibody comprising the light chain variable domain consisting of the sequence SEQ ID No. 10 above mentioned also comprises a heavy chain variable domain having the CDR-L1, CDR-L2 and CDR-L3 of sequences SEQ ID Nos. 1, 2 and 3, respectively.

In another embodiment of the invention, the humanized antibody consists of a humanized antibody, or an antigen binding fragment thereof, comprising a heavy chain variable domain of sequence SEQ ID No. 9 and a light chain variable domain of sequence SEQ ID No. 10.

In a particular aspect, the humanized antibody herein described is capable of binding to ADAM17 through a single domain, contrary to the antibody described in the prior art. In other words, the humanized antibody according to the invention is capable of binding to a single epitope whereas antibodies of the prior art bind to a conformational epitope composed of two different separated domains, the disintegrin-cysteine rich domain and the catalytic domain.

This property is of particular interest as the binding of the humanized antibody is not dependant to a particular conformation of ADAM17 according to the nature of the patient, the nature of the condition to be treated, the general state of the patient, etc. . . .

In an embodiment, it is described a humanized antibody, or an antigen-binding fragment thereof, that binds to an ADAM17 epitope and that inhibits the shedding of at least one substrate of ADAM17 wherein the said ADAM17 epitope consists of an epitope comprised within the membrane proximal domain (MPD) of sequence SEQ ID No. 25.

An antibody binding to the MPD of ADAM17 can be obtained by any of a number of techniques well known to those skilled in the art, including but not limited to, immunisation and hybridoma generation, monoclonal B-cell selection, phage display, ribosomal display, yeast display, expressed immune response sequencing coupled with targeted gene synthesis. Each process can be performed with the ADAM17 protein or a selected sub domain as the target antigen. One skilled in the art could select for MPD binding antibodies from a population of antibodies binding to the ADAM17 extracellular domain, or more particularly the MPD. Subsequent selection and characterisation may in also represent the selective step for MPD binding whereby all binders to the ADAM17 extracellular domain are selectively screened for binding to the MPD or selected sub domains of the MPD. Alternatively all selection steps may be performed against the MPD or selected sub domains of the MPD and binding to the native ADAM17 extracellular domain being employed as a subsequent selection and characterisation step.

According to a particular embodiment, the humanized antibody, or an antigen-binding fragment thereof, is characterized in that it consists of a monoclonal antibody.

“Monoclonal antibody” is understood to mean an antibody arising from a nearly homogeneous antibody population. More particularly, the individual antibodies of a population are identical except for a few possible naturally-occurring mutations which can be found in minimal proportions. In other words, a monoclonal antibody consists of a homogeneous antibody arising from the growth of a single cell clone (for example a hybridoma, a eukaryotic host cell transfected with a DNA molecule coding for the homogeneous antibody, a prokaryotic host cell transfected with a DNA molecule coding for the homogeneous antibody, etc.) and is generally characterized by heavy chains of one and only one class and subclass, and light chains of only one type. Monoclonal antibodies are highly specific and are directed against a single antigen.

By “binding”, “binds”, or the like, it is intended that the humanized antibody, or antigen binding fragment thereof, forms a complex with an antigen that is relatively stable under physiologic conditions. Methods for determining whether two molecules bind are well known in the art and include, for example, equilibrium dialysis, surface plasmon resonance, and the like. For the avoidance of doubt, it does not mean that the said humanized antibody or antigen-binding fragment could not bind or interfere, at a low level, to another antigen. As a preferred embodiment, the said humanized antibody, or antigen-binding fragment thereof, binds to its antigen with an affinity that is at least two-fold greater than its affinity for binding to a non specific molecule (BSA, casein, etc.). Nevertheless, as another preferred embodiment, the said humanized antibody, or antigen-binding fragment thereof, binds only to the said antigen.

For more clarity, table 2 below summarizes the various amino acid sequences corresponding to the humanized antibody of the invention.

TABLE 2

| | CDR | | | SEQ |
|---|---|---|---|---|
| Antibody | numbering | Heavy chain | Light chain | ID NO. |
| hz1022C3 | IMGT | CDR-H1 | | 1 |
| | | CDR-H2 | | 2 |
| | | CDR-H3 | | 3 |
| | | | CDR-L1 | 4 |
| | | | CDR-L2 | 5 |
| | | | CDR-L3 | 6 |
| | | Variable domain | | 7 |
| | | consensus | | |
| | | | Variable domain | 8 |
| | | | consensus | |
| | | variable domain | | 9 |
| | | | variable domain | 10 |
| | | Full length IgG1 | | 11 |
| | | Full length IgG2 | | 12 |
| | | Full length IgG3 | | 13 |
| | | Full length IgG4 | | 14 |
| | | | Full length | 15 |

The Membrane proximal domain (MPD), as already mentioned, consists of the amino acid domain 564-671 of the human ADAM17 (of sequence SEQ ID No. 22). It can also be noticed here that the corresponding MPD for the murine ADAM17 is also comprised of the amino acids 564-671.

According to a particular aspect, the humanized antibody herein described does not bind to the Metalloprotease (MP) domain, said MP domain being comprised of amino acid 215-474 of sequence SEQ ID No. 22.

According to a particular aspect, the humanized antibody herein described does not bind to the Disintegrin (DI) domain, said DI domain being comprised of amino acid 475-563 of sequence SEQ ID No. 22.

This aspect is surprising as it has never been described, nor suggested, an antagonist, and more particularly an antibody, and still more particularly a humanized antibody, capable of decreasing or inhibiting the shedding of ADAM17 substrates without interfering with the catalytic domain of ADAM17, known to be responsible for the shedding activity. In other words, the humanized antibody herein described is capable of selectively decreasing or inhibiting the enzymatic activity of ADAM17 regarding at least one substrate in the specific context of a pathology, said pathology being cancer. An advantage of the humanized antibody herein described may rely on the fact that it seems to not inhibit the whole catalytic activity of ADAM17 as it does not bind to the catalytic domain, but it may be capable of decreasing or inhibiting the enzymatic activity of ADAM17 for all or part of its substrates.

In an embodiment, the humanized antibody described in the present application inhibits the cellular shedding of at least one substrate of ADAM17 with an IC₅₀ of 500 pM or less, preferentially 200 pM or less.

In the context of the invention, the expression “IC₅₀” refers to the concentration of an antibody in a dose response evaluation that is necessary to achieve half the maximal attainable inhibition. Such evaluation of the IC₅₀ can be made by substrate shedding from cells evaluation or Fluorescence Resonance Energy Transfer (FRET) peptide cleavage assay with recombinant protein.

In a preferred aspect, the humanized antibody is capable of inhibiting the cellular shedding of TNF-α (Tumor necrosis factor alpha), and more preferably with at least an IC₅₀ of 500 pM or less, preferentially 200 pM or less.

In a preferred aspect, the humanized antibody according to the invention is capable of inhibiting the cellular shedding of TGF-α (Transforming growth factor alpha), and more preferably with at least an IC₅₀ of 500 pM or less, preferentially 200 pM or less.

In a preferred aspect, the humanized antibody is capable of inhibiting the cellular shedding of amphiregulin (AREG), and more preferably with at least an IC₅₀ of 500 pM or less, preferentially 200 pM or less.

In a preferred aspect, the humanized antibody according to the invention is capable of inhibiting the cellular shedding of HB-EGF (Heparin-binding EGF-like growth factor), and more preferably with at least an IC₅₀ of 500 pM or les, preferentially 200 pM or less.

In an embodiment, the humanized antibody of the invention is characterized in that it inhibits the cellular shedding of at least one substrate of ADAM17 selected from TNF-α, TGF-α, AREG and HB-EGF.

In an embodiment, the humanized antibody of the invention is characterized in that it inhibits with at least an IC₅₀ of 500 pM or less, preferentially 200 pM or less, the cellular shedding of at least one substrate of ADAM17 selected from TNF-α, TGF-α, AREG and HB-EGF.

In an embodiment, the humanized antibody of the invention is characterized in that it inhibits with at least an IC₅₀ of 500 pM or less, preferentially 200 pM or less, the cellular shedding of TNF-α, TGF-α, AREG and HB-EGF.

According to another aspect, the humanized antibody is characterized in that it binds to an ADAM17 epitope with a Kd of about 10 nM or less, preferentially of about 5 nM or less, as determined by surface plasmon resonance (SPR).

In another embodiment, the humanized antibody is characterized in that it binds to an ADAM17 epitope with a Kd of about 10 nM or less, preferentially of about 5 nM or less, and more preferentially of about 2 nM or less, as determined by surface plasmon resonance (SPR).

“K_{d}” also referred to as “Kd” or “KD” refers to the dissociation constant of a particular antibody-antigen complex. K_{d}=K_{off}/Kₒₙ with K_{off} consisting in the off rate constant for dissociation of the antibody from an antibody-antigen complex and Kₒₙ consisting in the rate at which the antibody associates with the antigen (Chen Y. et al., 1999; J Mol Biol. 1999; 293(4):865-81)).

In an embodiment, the humanized antibody, or an antigen binding fragment thereof, according to the invention is characterized in that said antigen binding fragment is a F(ab), a F(ab′), a F(ab′)₂ or a scFv fragment. The “antigen binding fragments” can be selected, without limitation, in the group consisting of Fv, scFv (sc for single chain), Fab, F(ab′)₂, Fab′, or any fragment of which the half-life time would be increased by chemical modification, such as the addition of poly(alkylene) glycol such as poly(ethylene) glycol (“PEGylation”) (pegylated fragments called Fv-PEG, scFv-PEG, Fab-PEG, F(ab′)₂-PEG or Fab′-PEG) (“PEG” for Poly(Ethylene) Glycol), or by incorporation in a liposome, said fragments having at least one of the characteristic variable domain of the humanized antibody according to the invention. Preferably, said “antigen binding fragments” will be constituted or will comprise a partial sequence of the heavy or light variable chain of the humanized antibody from which they are derived, said partial sequence being sufficient to retain the same specificity of binding as the humanized antibody from which it is descended and a sufficient affinity, preferably at least equal to 1/100, in a more preferred manner to at least 1/10, of the affinity of the humanized antibody from which it is descended, with respect to the target.

The term “epitope” is a region of an antigen that is bound by an antibody. Epitopes may be defined as structural or functional. Functional epitopes are generally a subset of the structural epitopes and have those residues that directly contribute to the affinity of the interaction. Epitopes may also be conformational. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. For the avoidance of doubt, the expression “single epitope” does not necessarily means a linear epitope.

The humanized antibody, or an antigen binding fragment thereof, according to the invention is of the IgG1, IgG2, IgG3, or IgG4 isotype.

As a non limitative, embodiment, the humanized antibody, or any antigen binding fragment thereof, is of the IgG1 isotype. As an example, the said IgG1 isotype humanized antibody according to the invention comprises a heavy chain of sequence SEQ ID No. 11 and a light chain comprising the CDR-L1, CDR-L2 and CDR-L3 of sequences SEQ ID Nos. 4, 5 and 6, respectively. In a preferred embodiment, the said humanized antibody comprises a light chain variable domain of sequence SEQ ID No. 8 and more preferably a light chain variable domain of sequence SEQ ID No. 10, and still more preferably a light chain of sequence SEQ ID No. 15.

As another non limitative, embodiment, the humanized antibody, or any antigen binding fragment thereof, is of the IgG2 isotype. As an example, the said IgG2 isotype humanized antibody according to the invention comprises a heavy chain of sequence SEQ ID No. 12 and a light chain comprising the CDR-L1, CDR-L2 and CDR-L3 of sequences SEQ ID Nos. 4, 5 and 6, respectively. In a preferred embodiment, the said humanized antibody comprises a light chain variable domain of sequence SEQ ID No. 8 and more preferably a light chain variable domain of sequence SEQ ID No. 10, and still more preferably a light chain of sequence SEQ ID No. 15.

As another non limitative, embodiment, the humanized antibody, or any antigen binding fragment thereof, is of the IgG3 isotype. As an example, the said IgG3 isotype humanized antibody according to the invention comprises a heavy chain of sequence SEQ ID No. 13 and a light chain comprising the CDR-L1, CDR-L2 and CDR-L3 of sequences SEQ ID Nos. 4, 5 and 6, respectively. In a preferred embodiment, the said humanized antibody comprises a light chain variable domain of sequence SEQ ID No. 8 and more preferably a light chain variable domain of sequence SEQ ID No. 10, and still more preferably a light chain of sequence SEQ ID No. 15.

As another non limitative, embodiment, the humanized antibody, or any antigen binding fragment thereof, is of the IgG4 isotype. As an example, the said IgG4 isotype humanized antibody according to the invention comprises a heavy chain of sequence SEQ ID No. 14 and a light chain comprising the CDR-L1, CDR-L2 and CDR-L3 of sequences SEQ ID Nos. 4, 5 and 6, respectively. In a preferred embodiment, the said humanized antibody comprises a light chain variable domain of sequence SEQ ID No. 8 and more preferably a light chain variable domain of sequence SEQ ID No. 10, and still more preferably a light chain of sequence SEQ ID No. 15.

In other words, the invention relates to a humanized antibody, or an antigen binding fragment thereof characterized in that it comprises a heavy chain of sequence selected from sequences SEQ ID No. 11, 12, 13, and 14 and a light chain of sequence SEQ ID No. 15.

An object of the scope of the present invention an affinity matured mutant of the humanized antibody above described.

In a preferred embodiment, the said affinity matured mutant consists of a mutant having higher affinity as compared to the said initial humanized antibody.

Any method known by the person skilled in the art should be used for affinity maturation. As non limitative example, it can be mentioned targeted or random mutagenesis of the variable domains, targeted or random mutagenesis of the CDR(s), chain shuffling with antibody libraries or novel heavy or light chains, cellular amelioration or other similarly appropriate methods followed by selection and screening for clones of higher affinity.

The invention relates to a humanized antibody, or an antigen binding fragment thereof, characterized in that it consists of an affinity matured mutant of the humanized antibody above described.

It is described a monoclonal antibody, or an antigen-binding fragment thereof, characterized in that it consists of the monoclonal antibody 1022C3 obtained from the hybridoma 1-4686 deposited at the CNCM, Institut Pasteur, 25 Rue du Docteur Roux, 75725 Paris Cedex 15, France, on the 18 Oct. 2012.

The invention relates to a humanized antibody, or an antigen binding fragment thereof, which is derived from the parental antibody consisting of the murine antibody 1022C3 obtained from the hybridoma 1-4686 deposited at the CNCM, Institut Pasteur, 25 Rue du Docteur Roux, 75725 Paris Cedex 15, France, on the 18 Oct. 2012. Said hybridoma was obtained by the fusion of Balb/C immunized mice splenocytes and cells of the myeloma Sp 2/O-Ag 14 lines.

It is also an object of the invention to claim a murine, chimeric, humanized or human ADAM17 antibody, or an antigen-binding fragment thereof, comprising:
i) the amino acid sequence of the heavy chain domain of the antibody expressed by the hybridoma cell line 1-4686 deposited at the CNCM; and
ii) the amino acid sequence of the light chain domain of the antibody expressed by the hybridoma cell line 1-4686 deposited at the CNCM.

In a particular embodiment, the humanized antibody herein described is capable of binding to an epitope of ADAM17 comprised within the membrane proximal domain (MPD) of sequence SEQ ID No. 25.

Another preferred property of the humanized antibody is that it inhibits the cellular shedding of at least one substrate of ADAM17 with an IC₅₀ of 500 pM or less, preferentially 200 pM or less, said at least one substrate of ADAM17 being preferentially selected from TNF-α, TGF-α, AREG and/or HB-EGF.

In another embodiment, the humanized antibody is characterized in that it binds to an ADAM17 epitope with a Kd of about 10 nM or less, preferentially of about 5 nM or less, and more preferentially of about 2 nM or less, as determined by surface plasmon resonance (SPR).

Another aspect of the invention is a method of inhibiting the growth and/or the proliferation of tumour cells wherein said method comprises a step of administering, to a patient in need thereof, an effective amount of the humanized antibody, or an antigen-binding fragment thereof, as above described.

In another embodiment, it is described a humanized antibody for use in a method of inhibiting the growth and/or the proliferation of tumour cells, said tumour cells being selected from tumour cells expressing ADAM17.

The person skilled in the art would easily determine the expression level of ADAM17 by any known technique such as cytometry, immunohistochemistry, Antibody Binding Capacity (ABC), etc. . . .

As a non limitative example, the expression level can be determined by measuring by cytometry the Antibody Binding Capacity (ABC) of a labelled antibody to ADAM17.

In an embodiment, the tumour cell is considered as expressing ADAM17 with an ABC of at least 5000.

In another embodiment, the tumour cell is considered as expressing ADAM17 with an ABC of at least 10000.

The invention consists also of the humanized antibody, or an antigen binding fragment thereof, as above described, for use as a medicament.

The invention also relates to a composition comprising as an active ingredient a humanized antibody, or an antigen binding fragment thereof, as described in the present specification. Preferably, said humanized antibody is supplemented by an excipient and/or a pharmaceutically acceptable carrier.

The invention also comprises the composition herein described for use as a medicament.

The present invention relates also to a humanized antibody, or antigen binding fragment thereof, or to a composition comprising such a humanized antibody, for its use as a medicament, particularly for the treatment of cancer.

The present invention is also directed to a method for treating cancer in a subject in need thereof comprising administering to said patient a therapeutically effective amount of at least a humanized antibody, or an antigen binding fragment thereof, or a composition comprising such a humanized antibody, thereby treating cancer in said subject.

The present invention also relates to the use of a humanized antibody, or an antigen binding fragment thereof, or of a composition comprising such a humanized antibody, for the preparation of a drug for inhibiting the growth of tumor cells expressing ADAM 17.

The present invention also relates to the use of a humanized antibody, or an antigen binding fragment thereof, or of a composition comprising such a humanized antibody, for the preparation of a drug for inhibiting the growth of tumor cells over-expressing ADAM 17.

The present invention is also directed to a method for treating a disease related to tumor cells proliferation in a subject in need thereof comprising administering to said patient a therapeutically effective amount of at least a humanized antibody, or an antigen binding fragment thereof, or a composition comprising such a humanized antibody, wherein said humanized antibody is capable of inhibiting the growth of tumor cells, thereby treating said disease in said subject.

In a preferred manner, said cancer is a cancer chosen among estrogen-related breast cancer, non-small cell lung cancer, colon cancer and/or pancreatic cancer.

In other words, the invention relates to the humanized antibody, or an antigen binding fragment thereof, or the composition comprising such a humanized antibody, for its use as a medicament for the treatment of cancer.

In a preferred embodiment, said cancer is a cancer selected among prostate cancer, lung cancer, breast cancer, ovarian cancer, pancreatic cancer, head and neck cancer, cancer of the kidney and colon cancer.

Another aspect of the present invention relates to an isolated nucleic acid coding for a humanized antibody, or an antigen binding fragment thereof, as described.

The terms “nucleic acid”, “nucleic sequence”, “nucleic acid sequence”, “polynucleotide”, “oligonucleotide”, “polynucleotide sequence” and “nucleotide sequence”, used interchangeably in the present description, mean a precise sequence of nucleotides, modified or not, defining a fragment or a region of a nucleic acid, containing unnatural nucleotides or not, and being either a double-strand DNA, a single-strand DNA or transcription products of said DNAs.

It should also be included here that the present invention does not relate to nucleotide sequences in their natural chromosomal environment, i.e., in a natural state. The sequences of the present invention have been isolated and/or purified, i.e., they were sampled directly or indirectly, for example by a copy, their environment having been at least partially modified. Isolated nucleic acids obtained by recombinant genetics, by means, for example, of host cells, or obtained by chemical synthesis should also be mentioned here.

An embodiment of the invention consists of an isolated nucleic acid coding for the humanized antibody of the invention, characterized in that it comprises a heavy chain variable domain comprising the sequence SEQ ID No. 16 and a light chain variable domain comprising the sequence SEQ ID No. 17.

It is also described a vector comprising a nucleic acid as above mentioned.

The invention notably targets cloning and/or expression vectors that contain such a nucleotide sequence.

The vector of the invention preferably contains elements which allow the transcription/translation expression and/or the secretion of nucleotide sequences in a given host cell, resulting in the expression/secretion of the encoded humanized antibody. The vector thus must contain a promoter, translation initiation and termination signals, as well as suitable transcription regulation regions. In a preferred embodiment, such vector should be able to be maintained in a stable manner in the host cell and may optionally have specific signals which specify secretion of the translated antibody. These various elements are selected and optimized by a person skilled in the art according to the host cell used. For this purpose, the nucleotide sequences can be inserted in self-replicating vectors within the chosen host or be integrative vectors of the chosen host.

Such vectors are prepared by methods typically used by a person skilled in the art and the resulting clones can be introduced into a suitable host by standard methods such as lipofection, electroporation, heat shock or chemical methods.

The vectors are, for example, vectors of plasmid or viral origin. They are used to transform host cells in order to clone or express the nucleotide sequences of the invention.

The invention also relates to a host cell comprising a vector as above described.

The host cell can be selected among prokaryotic or eukaryotic systems such as bacterial cells, for example, but also yeast cells or animal cells, notably mammal cells. Insect or plant cells can also be used.

Another topic of the present application consists of a transgenic animal with the exception of human comprising at least one cell transformed by the humanized antibody as above described.

It is thus also described a process for production of the humanized antibody, or an antigen-binding fragment thereof, according to the present specification, characterized in that it comprises the following stages:

a) the culture in a medium and appropriate culture conditions of a cell as above described; and

b) the recovery of said antibody, or antigen-binding fragment thereof, thus produced starting from the culture medium or said cultured cells; and

c) the humanization of said antibody.

The transformed cells according to the invention are of use in methods for the preparation of recombinant polypeptides according to the invention. Methods for the preparation of polypeptide according to the invention in recombinant form, characterized in that said methods use a vector and/or a cell transformed by a vector according to the invention, are also comprised in the present invention. Preferably, a cell transformed by a vector according to the invention is cultured under conditions that allow the expression of the aforesaid polypeptide and recovery of said recombinant peptide.

As already mentioned, the host cell can be selected among prokaryotic or eukaryotic systems. In particular, it is possible to identify the nucleotide sequences of the invention that facilitate secretion in such a prokaryotic or eukaryotic system. A vector according to the invention carrying such a sequence can thus be used advantageously for the production of recombinant proteins to be secreted. Indeed, the purification of these recombinant proteins of interest will be facilitated by the fact that they are present in the supernatant of the cellular culture rather than inside host cells.

The polypeptides of the invention can also be prepared by chemical synthesis. One such method of preparation is also an object of the invention. A person skilled in the art knows methods for chemical synthesis, such as solid-phase techniques (see notably Steward et al., 1984, Solid phase peptides synthesis, Pierce Chem. Company, Rockford, 111, 2nd ed.) or partial solid-phase techniques, by condensation of fragments or by conventional synthesis in solution. Polypeptides obtained by chemical synthesis and capable of containing corresponding unnatural amino acids are also comprised in the invention.

The humanized antibody, or an antigen binding-fragment thereof, likely to be obtained by the method of the invention is also comprised in the present invention.

The present invention relates to the humanized antibody described above, or an antigen-binding fragment thereof, for use as a medicament.

In an embodiment, the present application relates to a composition comprising the humanized antibody as above described.

In an embodiment, the composition is characterized in that it further comprises, as a combination product, a therapeutically effective amount of a cytotoxic agent.

The constituents of which the combination is composed may be administered simultaneously, separately, or sequentially so as to obtain the maximum efficacy of the combination; it being possible for each administration to vary in its duration from a rapid administration to a continuous perfusion.

As used herein, “simultaneous administration” refers to the administration of the two compounds of the composition according in a single and unique pharmaceutical form.

As used herein, “separate administration” refers to the administration, at the same time, of the two compounds of the composition according to the invention in distinct pharmaceutical forms.

As used herein, “sequential administration” refers to the successive administration of the two compounds of the composition according to the invention, each in a distinct pharmaceutical form.

A “therapeutically effective amount”, as used herein, refers to the minimum concentration or amount of a compound (or of compounds) which is effective to prevent, alleviate, reduce or ameliorate symptoms of disease or prolong the survival of the patient being treated. A therapeutically effective amount is also one in which any toxic or detrimental effects of the agent are outweighed by the therapeutically beneficial effects. More particularly, in reference to the treatment of cancer, a therapeutically effective amount refers to that amount which has the effect of (1) reducing the size of (or preferably eliminating) the tumour; (2) inhibiting (that is, slowing to some extent, preferably stopping) tumour metastasis; (3) inhibiting to some extent (that is slowing to some extent, preferably stopping) tumour growth; and/or, (4) relieving to some extent (or preferably eliminating) one or more symptoms associated with the cancer.

In an embodiment, the composition is characterized in that it further comprises, as a conjugation product, a therapeutically effective amount of a cytotoxic agent.

In the sense of the present invention, the expression “conjugation” refers generally to a composition comprising at least the humanized antibody above described physically linked with a one or more cytotoxic agent(s), thus creating a highly targeted compound.

In an embodiment, the humanized antibody is chemically linked to the cytotoxic agent by a linker. “Linker”, “Linker Unit”, or “link” means a chemical moiety comprising a covalent bond or a chain of atoms that covalently attaches a binding protein to at least one cytotoxic agent. The linker may be a “non cleavable” or “cleavable”.

The composition of the invention may also contain various diluents, fillers, salts, buffers, stabilizers, solubilizers, and other materials well known in the art.

In another embodiment, the composition is a pharmaceutical composition further comprising a pharmaceutically acceptable vehicle.

As used herein, “pharmaceutically acceptable vehicle” or “pharmaceutically acceptable carrier” includes any and all solvents, buffers, salt solutions, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, topical, transdermal or oral administration. Pharmaceutically acceptable carriers include sterile aqueous solutions or dispersions and sterile powders for the extemporaneous preparation of sterile injectable solutions or dispersion. The use of media and agents for pharmaceutically active substances is well known in the art. Methods for preparing parenterally administrable compounds will be known or apparent to those skilled in the art and are described in more detail in for example, Remington's Pharmaceutical Science, 17th ed., Mack Publishing Company, Easton, Pa. (1985), and the 18^{th} and 19^{th} editions thereof, which are incorporated herein by reference.

By “cytotoxic agent” or “cytotoxic”, it is intended an agent which, when administered to a subject, treats or prevents the development of cell proliferation, preferably the development of cancer in the subject's body, by inhibiting or preventing a cellular function and/or causing cell death.

Many cytotoxic agents have been isolated or synthesized and make it possible to inhibit the cells proliferation, or to destroy or reduce, if not definitively, at least significantly the tumour cells.

More particularly, the cytotoxic agent may preferably consist of, without limitation, a drug, a toxin, a radioisotope, etc.

According to another aspect, it is described the composition for use in a method of inhibiting the growth and/or the proliferation of tumour cells.

The present invention also comprises the use of the composition for the preparation of a medicament, said medicament being preferably intended for the prevention or the treatment of cancer.

For therapeutic applications, the composition of the invention is administered to a mammal, preferably a human, in a pharmaceutically acceptable dosage form such as those discussed above, including those that may be administered to a human intravenously as a bolus or by continuous infusion over a period of time, by intramuscular, intraperitoneal, intracerebrospinal, subcutaneous, intraarticular, intrasynovial, intrathecal, oral, topical, or inhalation routes. The said composition of the invention is also suitably administered by intratumoural, peritumoural, intralesional, or perilesional routes, to exert local as well as systemic therapeutic effects.

Other characteristics and advantages of the invention appear further in the description with the examples and figures whose legends are presented below.

### LEGEND OF THE FIGURES

FIGS. 1A, B and C: SDS-PAGE Analysis of: 1A) murine 1022C3, 1B) humanised 1022C3 variants (Vn) with germlines IGHV2-5 and IGKV1-33, 1C) humanised 1022C3 variants (Vn) with germlines IGHV4-4 and IGKV1-39 and finalised variant hz1022C3.

FIGS. 2A, B and C: Comparison of m1022C3 and hz1022C3 maximal inhibition of shedding of Nluc fusion proteins AREG (A: Means are significantly different: Unpaired t test (a<0.05): p=0.0005), TGFα (B: Means are significantly different: Unpaired t test (a<0.05): p=0.0003) and HB-EGF (C: Means are significantly different: Unpaired t test (a<0.05): p=0.0069) from stably transfected cell line A431 expressed as a percentage of baseline shedding levels of untreated cells.

FIG. 3: Peptide micro array epitope characterization, hybridization signal ratio of 1022C3 and ch9G4 to membrane proximal domain over lapping peptide array.

FIG. 4: In vivo activity of the 1022C3 on established A431 xenografts.

FIG. 5: In vivo activity of the 1022C3 on a fragment A431 xenograft model.

FIG. 6: Comparison of m1022C3 (murine form) and c1022C3 (chimeric form) on the A431 xenograft model.

FIG. 7: In vivo activity of the 1022C3 on established CaOV-3 xenografts

FIG. 8: In vivo activity of the 1022C3 on established OVISE xenografts.

FIG. 9: In vivo activity if the 1022C3 on established BxPC3 xenografts.

FIGS. 10A et B: Comparison of the murine 1022C3 (m1022C3) with its humanized form (hz1022C3) on the CaOV3 xenograft model when used at 1.25 mg/kg (FIG. 10A) and when used at 5 mg/kg (FIG. 10B).

### EXAMPLE 1: GENERATION OF THE MURINE ANTIBODY

To generate murine monoclonal antibodies (mAbs) against human ADAM17, 5 BALB/c mice were immunized 3-times sub cutaneously with 15-20 μg of the human ADAM17 recombinant protein (R and D Systems, ref: 930-ADB, rhADAM17). The first immunization was performed in the presence of Complete Freund's Adjuvant (Sigma, St Louis, Md., USA). Incomplete Freund's adjuvant (Sigma) was added for following immunizations.

Three days prior to the fusion, 2 immunized mice (selected based on sera titration) were boosted with 15-20 μg of rhADAM17 protein with incomplete Freund's adjuvant. Lymphocytes were prepared by mincing of the proximal lymph nodes, they were then fused to 5P2/0-Ag14 myeloma cells in a 1:4 ratio (lymphocyte:myeloma) (ATCC, Rockville, Md., USA). The fusion protocol is that described by Kohler and Milstein (1975), finally, 50 96 well plates were seeded. Fused cells were then subjected to metabolic HAT selection. Approximately 10 days after the fusion, colonies of hybrid cells were screened. For the primary screen, supernatants of hybridomas were evaluated for the secretion of mAbs raised against human ADAM17 using an ELISA.

Briefly, 96-well ELISA plates (Costar 3690, Corning, N.Y., USA) were coated with 50 μl/well of the recombinant human ADAM17 protein (R and D Systems, ref: 930 ADB) at 0.7 μg/ml in PBS overnight at 4° C. The plates were then blocked with PBS containing 0.5% gelatin (#22151, Serva Electrophoresis GmbH, Heidelberg, Germany) for 2 h at 37° C. Once the saturation buffer discarded by flicking plates, 50 μl of sample (hybridoma supernatant or purified antibody) was added to the ELISA plates and incubated for 1 h at 37° C. After three washes, 50 μl horseradish peroxidase-conjugated polyclonal goat anti-mouse IgG (#115-035-164, Jackson Immuno-Research Laboratories, Inc., West Grove, Pa., USA) was added at a 1/5000 dilution in PBS containing 0.1% gelatin and 0.05% Tween 20 (w:w) for 1 h at 37° C. ELISA plates were washed 3-times and TMB (#UP664782, Uptima, Interchim, France) substrate was added. After a 10 min incubation time at room temperature, the reaction was stopped using 1 M sulfuric acid and the optical density at 450 nm was measured.

As a second screening step, selected hybridoma supernatants were evaluated by FACS analysis for mAbs able to bind the cellular form of ADAM17 expressed on the surface of A172 human tumour cells. For the selection by flow cytometry, 2×10⁵ cells were plated in each well of a 96 well-plate in PBS containing 1% BSA and 0.01% sodium azide (FACS buffer) at 4° C. After a 2 min centrifugation at 2000 rpm, the buffer was removed and hybridoma supernatants to be tested were added. After 20 min of incubation at 4° C., cells were washed twice and an Alexa 488-conjugated goat anti-mouse antibody diluted 1/500 in FACS buffer (#A11017, Molecular Probes Inc., Eugene, USA) was added and incubated for 20 min at 4° C. After a final wash with FACS buffer, cells were analyzed by FACS (Facscalibur, Becton-Dickinson) after addition of propidium iodide to each tube at a final concentration of 40 μg/ml. Wells containing cells alone and cells incubated with the secondary Alexa 488-conjugated antibody were included as negative controls. Isotype controls were used in each experiment (Sigma, ref M90351MG). At least 5000 cells were assessed to calculate the mean value of fluorescence intensity (MFI).

As soon as possible, selected hybridomas were cloned by limiting dilution. One 96-well plate was prepared for each code. A volume of 100 μl of a cell suspension adjusted to 8 cells/ml in cloning specific culture medium was loaded in each well. At Day 7, the wells were microscopically examined to ensure cloning and plating efficiency before refeeding the plates with 100 μl of cloning specific culture medium. At days 9-10, the hybridoma supernatants were subsequently screened for their reactivity against the rhADAM17 protein. Cloned mAbs were then isotyped using an Isotyping kit (cat #5300.05, Southern Biotech, Birmingham, Ala., USA). One clone obtained from each hybridoma was selected and expanded to confirm their binding specificity against rhADAM17 and human tumour cells (A172).

### EXAMPLE 2: HUMANIZATION PROCESS

Humanized variants of the variable domains were generated by gene synthesis (Genecust, Luxembourg) and cloned in the episomal expression vector pCEP (Invitrogen). All cloning steps were performed according to conventional molecular biology techniques as described in the Laboratory manual (Sambrook and Russel, 2001) or according to the supplier's instructions. Each vector was validated by nucleotide sequencing using Big Dye terminator cycle sequencing kit (Applied Biosystems, US) and analyzed using a 3500 Genetic Analyzer (Applied Biosystems, US). For transient antibody expression 2.10⁶ cells/ml were transefected with two different vectors at a ratio of 1:1. One vector was coding for the antibody heavy chain and the other one for the light chain. Plasmid DNA (final concentration 1.25 μg/ml) was mixed with linear 25 kDa polyethyleneimine (PEI) (Polysciences) prepared in water at a final concentration of 1 mg/ml. The weight ratio DNA to PEI used for transfection was 1:4. For complex formation DNA and PEI were incubated for 10 min before adding to the cells. 4 hours after transfection cells were diluted in the same medium to a concentration of 1.10⁶ cells/ml and valproic acid sodium salt (Sigma-Ref. P4543-100G) was added at a final concentration of 0.156 nM. Expression volumes varied from approximately 7 ml in 50 ml bioreactors for screening to several hundreds of ml for medium scale expression. Antibodies concentration was determined using ForteBio biosensors. Antibodies were purified from the supernatant using protein A magnetic beads or protein A columns. Bound antibodies were eluted with Gly/HCl 0.1 M buffer at pH3 and then dialysis against 50 mM Tris-HCl pH 7.5 buffer. Purity and VH/VL assembly were validated using SDS-PAGE with coomasie blue staining. Protein aggregation was measured by size exclusion chromatography.

Inhibitory activities of the humanized variants were determined using recombinant ADAM-17 in a fluorescence peptide cleavage assay (InnoZyme™ TACE Activity Kit, EMD Millipore) or in cellular activity assay. In the cellular assay cells were transfected with a recombinant fusion of an appropriate ADAM-17 membrane bound substrate genetically fused to the reporter gene nanoluciferase (Promega). ADAM-17 substrates included TNF-alpha, TGF-beta, HB-EGF and amphiregulin. ADAM-17 activity was measured by the shedding of this fusion protein in the supernatant.

### EXAMPLE 3: ANTIBODY ASSEMBLY AND GERMLINE SELECTION

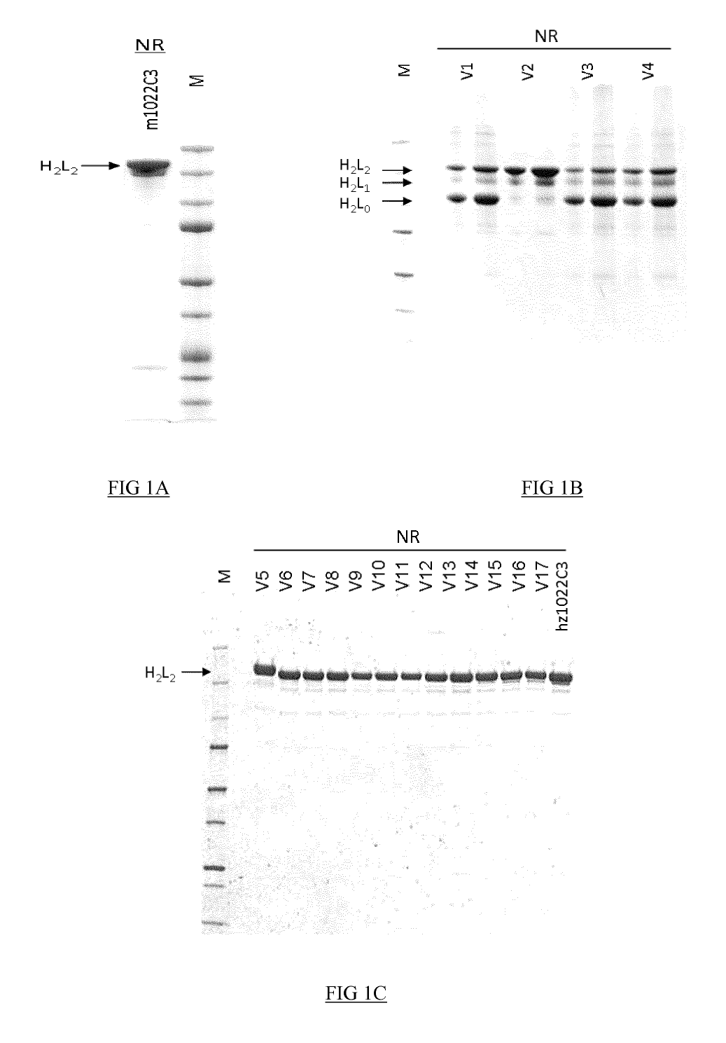

Expressed antibodies were adjusted to pH 7.5 and purified on protein A sepharose columns with elution by pH shift with Glycine, HCl 0.1 M, pH 3, the resulting eluant was adjusted to pH 7.5 by dialysis against Tris-HCl pH 7.5 solution. The protein solutions were normalised by total protein quantification using the bicinchoninic acid assay. Equal quantities of protein were separated by SDS-PAGE in non-reducing conditions, and visualised by GelCode Blue Safe Protein Stain (BioRad). Four humanised variants were analysed for germline combination IGHV2-5 and IGKV1-33, V1-4, and thirteen variants with germlines IGHV4-4 and IGKV1-39, V5-V17 and hz1022C3. The humanised variants based on germlines IGHV2-5 and IGKV1-33 demonstrate poorly assembled antibodies with H2L1 and H2L0 present at considerable levels (FIG. 1B). The parental m1022C3 and humanised variants based on germlines IGHV4-4 and IGKV1-39 demonstrate principally the correctly assembled H2L2 (FIGS. 1A & 1C).

### EXAMPLE 4: ADAM 17 SHEDDING OF RECOMBINANT SUBSTRATES FROM TUMOUR CELL LINE A431

Stably transfected A431 cell lines, expressing at their plasma membrane pro-TGFα, pro-HB-EGF or pro-amphiregulin (AREG) fused to NanoLuc® luciferase (Promega), were generated. ADAM17 activity at the plasma membrane of these cells resulted in the release in the culture medium of the mature ligands fused to NanoLuc® Luciferase. Time dependant measurements of NanoLuc® luciferase activity in culture medium samples reflected the ADAM17 activity. A431 substrate-Nluc cells were seeded at 30 000 cells/well in a 96 well culture plate. Two days later, culture medium was replaced by 200 μl of fresh culture medium in which was diluted different concentrations of the anti-ADAM17 mAb (hz1022C3) or the irrelevant mAb(9G4). After 24 h of culture (37° C., CO₂ 5%) 5 μl of culture medium in all experimental wells were collected and distributed in separate wells of a white half-area 96 well plate. After addition of 15 μl of (PBS, BSA 0.1% v/v) Nano-Glo™ luciferase substrate (furimazine), total luminescence for each experimental point was read during 0.1 s on a (Berthold Mithras LB940) multimode microplate reader.

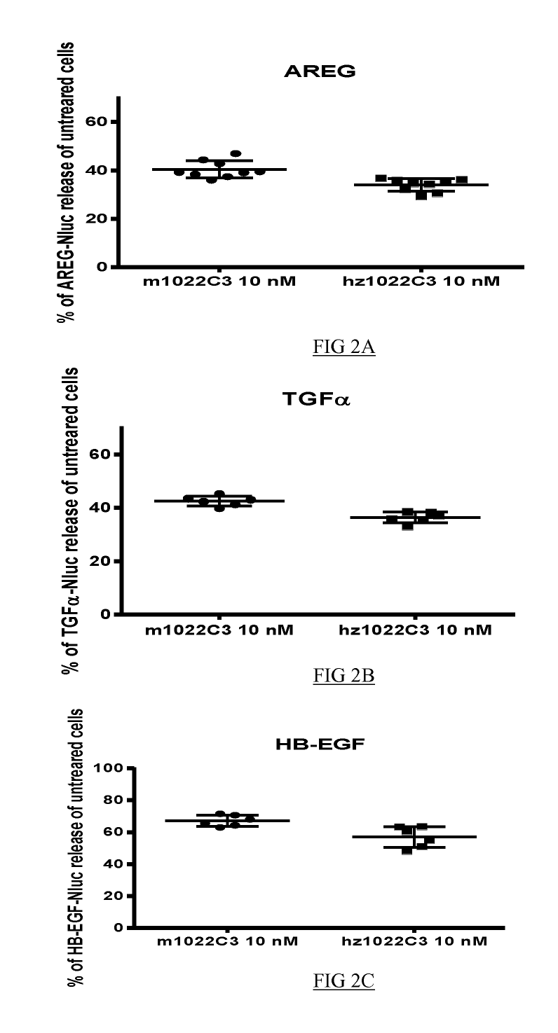

At a concentration giving the maximal inhibitory effect (10 nM), hz1022C3 showed a significantly increased inhibition of the release of AREG-Nluc, TGFα-Nluc or HB-EGF-Nluc than m1022C3 (+6.35% ±1.47%; +6.12% ±1.13% and +10.15% ±2.99% respectively) (FIGS. 2A-2C respectively).

The IC₅₀ values obtained for the inhibition of shedding of TGFα, pro-HB-EGF, pro-amphiregulin or a mutated pro-TNFα with mAb hz1022C3 were compared to those published in the literature (Table 3). The mAb hz1022C3 demonstrated significantly greater than 10 fold, preferably greater than 20 fold, preferably greater than 30 fold, preferably greater than 40 fold and preferably greater than 50 fold inhibition of the cellular shedding of all substrates tested compared to the reference compounds.

TABLE 3

| | IC₅₀ (nM) | | |
|---|---|---|---|
| | Inhibitor | | |
| Substrate | 1022C3* | D1(A12)** | N-TIMP-3** |
| TNFα | 0.16 | 11.2 | 48.5 |
| TGFα | 0.12 | 9.4 | 44.5 |
| AREG | 0.16 | 9.3 | 53.3 |
| HB-EGF | 0.19 | 7.9 | 47.3 |
| mean | 0.16 | 9.6 | 48.6 |

*Results obtained from at least 6 independent experiments
**Results obtained from patent application WO2012/104581 p40-41 table S1

### EXAMPLE 5: DEFINITION OF THE DISSOCIATION CONSTANT OF THE BINDING OF THE EXTRACELLULAR DOMAIN OF ADAM-17 ON MONOCLONAL ANTIBODY HZ1022C3 IGG1 AND IGG4 WITH LABEL FREE REAL-TIME SPR SINGLE CYCLE KINETIC EXPERIMENTS

Each antibody (used as the ligand) was bound at two different level on the second flowcell of a Biacore CM5 sensor chip (GE Healthcare) activated on both flowcells with a mouse anti-human IgG Fc specific covalently linked to the carboxymethyldextran matrix. For each antibody and each level of capture, five concentrations of soluble ADAM17 (the analyte with a molecular weight of 52 kDa) at concentrations ranging either from 12.5 to 200 nM or from 6.25 to 100 nM obtained by a two fold dilution scheme were injected in the growing range of concentrations at a flow rate of 30 μl/min. Each concentration was injected during 90 s. At the end of the 5^{th} injection, the running buffer was injected during 600 s for the dissociation phase measurement. At the end of each cycle, the surface was regenerated by an injection of 10 mM Glycine, HCl pH 1.7 buffer during 30 s. The sensorgramms obtained were double referenced by first subtracting the signal from the reference FC1 surface (without any anti-ADAM17 antibodies) followed by subtraction of the sensorgramm obtained for each antibody and each level of capture by 5 injections of the running buffer (Biacore HBS-EP buffer). Data were processed using BIAevaluation 3.1 software using the 1:1 Langmuir model.

The results are summarized in the following table 4.

TABLE 4

| | capture level | [ADAM-17] | Rmax | kon | koff | Kd | |
|---|---|---|---|---|---|---|---|
| Antibodies | RU | range | (RU) | (1/M · s) | (1/s) | (nM) | Chi2 |
| Hz1022C3 IgG1 | 1152.6 ± 5.6 | [12.5-200 nM] | 635.60 | 1.53E+05 | 2.46E−04 | 1.61 | 4.26 |
| | | [6.25-100 nM] | 594.00 | 1.78E+05 | 2.42E−04 | 1.36 | 2.26 |
| | 452.0 ± 1.3 | [12.5-200 nM] | 246.40 | 2.27E+05 | 2.27E−04 | 1.00 | 1.57 |
| | | [6.25-100 nM] | 232.50 | 2.68E+05 | 2.24E−04 | 0.83 | 0.94 |
| | | | | | mean ± SE | 1.20 ± 0.35 nM | |
| Hz1022C3 IgG4 | 1171.0 ± 2.6 | [12.5-200 nM] | 643.10 | 1.32E+05 | 1.82E−04 | 1.38 | 4.01 |
| | | [6.25-100 nM] | 601.00 | 1.56E+05 | 1.82E−04 | 1.16 | 2.35 |
| | 480.5 ± 1.6 | [12.5-200 nM] | 263.50 | 1.94E+05 | 1.76E−04 | 0.91 | 2.28 |
| | | [6.25-100 nM] | 248.50 | 2.33E+05 | 1.77E−04 | 0.76 | 1.74 |

| | |
|---|---|
| mean ± SE | 1.05 ± 0.27 nM |

### EXAMPLE 6: PEPTIDE MICRO ARRAY EPITOPE CHARACTERIZATION

Peptide micro array epitope characterization was performed by JPT Peptide Technologies GmbH, Berlin, Germany. In brief the analysis was performed as follows: 32-linear 15-meric peptide sequences representing an overlapping peptide scan (15/12) through the ADAM17 membrane proximal domain were synthesized (Table 5) and immobilized on microarray slides four additional peptides were included by JPT as internal process control spots. Moreover, human IgG and mouse IgG were co-immobilized on the microarray to serve as assay controls. Furthermore, rhADAM17 protein was co-immobilized as an additional control.

The assay was performed using a Multiwell incubation chamber. Microarrays were incubated with 100 μg/ml 1022C3 or control antibody ch9G4 diluted in blocking buffer (Pierce International, Superblock TBS T20, order No 37536) overnight at 4° C. Microarrays were then incubated with 1 μg/ml Cy5 labelled secondary antibody (anti-human IgG, JIR, 109-175-098) diluted in blocking buffer for 60 min at 30° C. Microarrays were dried. Before each step microarrays were washed with washing buffer (50 mM TBS-buffer, 0.1% Tween20 (v/v), pH 7.2).

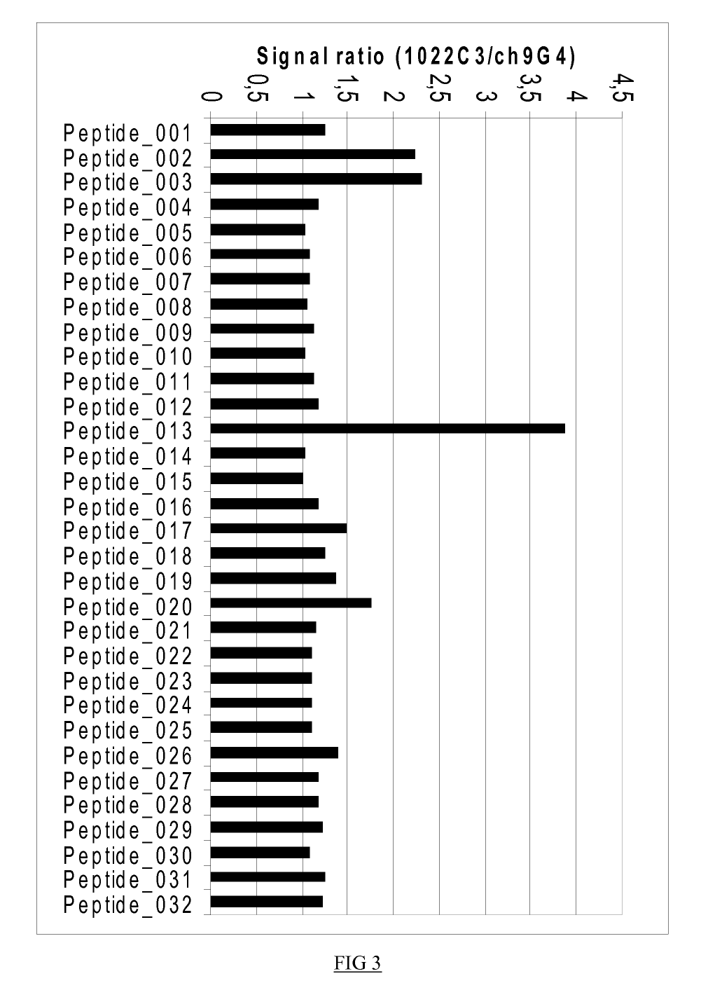

Microarrays were scanned using a high resolution fluorescence scanner. Laser settings and applied resolution were identical for all performed measurements. The resulting images were analyzed and quantified using spot-recognition software GenePix (Molecular Devices). For each spot, the mean signal intensity was extracted (between 0 and 65535 arbitrary units). The ration of signal intensity between 1022C3 and the control antibody ch9G4 was compared between multiple experiments and peptides with a signal three fold higher 1022C3 than ch9G4 were identified (FIG. 3).

TABLE 5

| Sequence | Name | SEQ ID No. |
|---|---|---|
| DTVCLDLGKCKDGKC | Peptide_001 | 26 |
| CLDLGKCKDGKCIPF | Peptide_002 | 27 |
| LGKCKDGKCIPFCER | Peptide_003 | 28 |
| CKDGKCIPFCEREQQ | Peptide_004 | 29 |
| GKCIPFCEREQQLES | Peptide_005 | 30 |
| IPFCEREQQLESCAC | Peptide_006 | 31 |
| CEREQQLESCACNET | Peptide_007 | 32 |
| EQQLESCACNETDNS | Peptide_008 | 33 |
| LESCACNETDNSCKV | Peptide_009 | 34 |
| CACNETDNSCKVCCR | Peptide_010 | 35 |
| NETDNSCKVCCRDLS | Peptide_011 | 36 |
| DNSCKVCCRDLSGRC | Peptide_012 | 37 |
| CKVCCRDLSGRCVPY | Peptide_013 | 38 |
| CCRDLSGRCVPYVDA | Peptide_014 | 39 |
| DLSGRCVPYVDAEQK | Peptide_015 | 40 |
| GRCVPYVDAEQKNLF | Peptide_016 | 41 |
| VPYVDAEQKNLFLRK | Peptide_017 | 42 |
| VDAEQKNLFLRKGKP | Peptide_018 | 43 |
| EQKNLFLRKGKPCTV | Peptide_019 | 44 |
| NLFLRKGKPCTVGFC | Peptide_020 | 45 |
| LRKGKPCTVGFCDMN | Peptide_021 | 46 |
| GKPCTVGFCDMNGKC | Peptide_022 | 47 |
| CTVGFCDMNGKCEKR | Peptide_023 | 48 |
| GFCDMNGKCEKRVQD | Peptide_024 | 49 |
| DMNGKCEKRVQDVIE | Peptide_025 | 50 |
| GKCEKRVQDVIERFW | Peptide_026 | 51 |
| EKRVQDVIERFWDFI | Peptide_027 | 52 |
| VQDVIERFWDFIDQL | Peptide_028 | 53 |
| VIERFWDFIDQLSIN | Peptide_029 | 54 |
| RFWDFIDQLSINTFG | Peptide_030 | 55 |
| DFIDQLSINTFGKFL | Peptide_031 | 56 |
| IDQLSINTFGKFLAD | Peptide_032 | 57 |

### EXAMPLE 7: IN VIVO EVALUATION OF THE MAB 1022C3

For all in vivo evaluations, six to eight weeks old athymic mice were used. They were housed in sterilized filter-topped cages, maintained in sterile conditions and manipulated according to French and European guidelines.

ADAM17 expression levels were determined by staining, 1×10⁵ cells/100 μl in FACS buffer (PBS containing 1% BSA and 0.01% sodium azide) incubated for 20 min. at 4° C. with increasing concentrations of the MAB9301 (Clone 111633, R&D systems) in order to determine a saturating concentration. Cells were then washed three times in FACS buffer. Cells were resuspended and incubated for 20 min. at 4° C. with a goat anti-mouse IgG-Alexa 488 antibody (Invitrogen Corporation, Scotland, # A11017). Cells were then washed three times in FACS buffer. Labelled cells were then resuspended in 100 μl of FACS buffer prior to analysis with a Facscalibur cytometer (Becton Dickinson, Le Pont-de-Claix, France). Propidium iodide was added to analyse only viable cells. In parallel, QIFIKIT beads were used for the determination of antibody-binding and antigen density per cell by flow cytometry and monoclonal antibody binding. QIFIKIT contains a series of beads, 10 μm in diameter and coated with different, but well-defined quantities of mouse mAb molecules. The beads mimic cells with different antigen densities which have been labelled with a primary mouse mAb. The quantified antigen is expressed in Antibody-Binding Capacity (ABC) units.

### 7.1 A431 Xenograft Model: Established Tumours

A431, an epidermoid carcinoma cell line, expressing ADAM17 (ABC=17 000), was selected for in vivo evaluations. Mice were injected subcutaneously at D0 with 10×10⁶ cells. When tumours reached approximately 200 mm³ (20 days post tumour cell injection), animals were divided into two groups of 6 mice with comparable tumour size and treated intraperitoneally with a loading dose of 20 mg/kg and then weekly with maintenance doses of 10 mg/kg of m1022C3 monoclonal antibody. A control group received only the vehicle as previous experiments performed in this model demonstrated that no difference in tumour growth was observed between mice treated with vehicle and mice injected with an isotype control. The mice were followed for the observation of xenograft growth rate. Tumour volume was calculated by the formula: π/6×length×width×height.

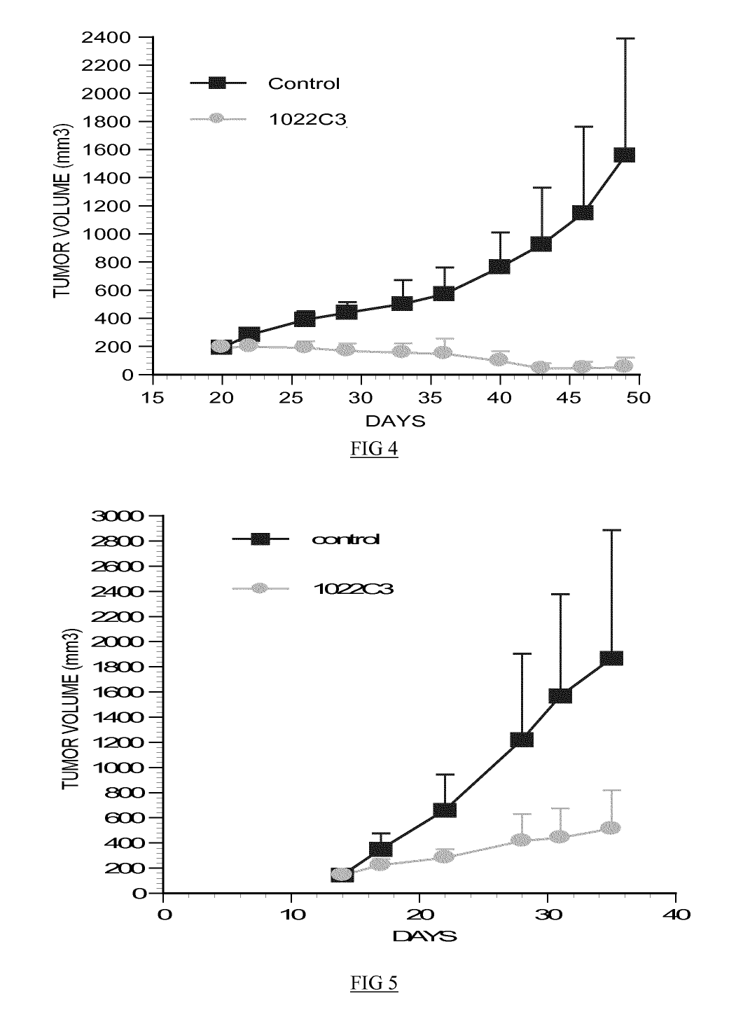

The results obtained were summarized in FIG. 4. They showed a dramatic tumour inhibition (97% at D49) mediated by the m1022C3 mAb with tumour regressions observed in all treated mice and complete regressions achieved in 2 out of 6 mice at D49.

### 7.2 A431 Xenograft Model Established with Tumour Fragments

In order to determine the robustness of the observed antitumour activity of the mAb m1022C3, tumours were first generated by cell implantation as described above. Then when tumour volume reached approximately 200-300 mm³, tumours were removed aseptically, minced in 1 mm³ fragments, carefully avoiding necrotic areas, and these fragments were then used for tumour propagation in a new series of athymic mice. This propagation cycle was performed 3 times in order to stabilize both tumour growth and characteristics and then 2 groups of 6 mice bearing tumours reaching approximately 145 mm³ (14 days post tumour cell injection), were generated. One group was treated intraperitoneally with a loading dose of 20 mg/kg and then weekly with maintenance doses of 10 mg/kg of m1022C3 monoclonal antibody. The second group received only the vehicle. The mice were followed for the observation of xenograft growth rate. Tumour volume was calculated by the formula: π/6×length×width×height.

The results obtained were summarized in FIG. 5. Tumours established from fragments grow faster than those resulting from cell engraftment. This observation was in agreement with a more aggressive phenotype of these tumours. In this more aggressive setting the m1022C3 is still significantly active with a tumour inhibition reaching 73% at day 35.

### 7.3 Comparison of the Murine 1022C3 (m1022C3) with its IgG1 Chimeric Form (c1022C3) on the A431 Xenograft Model

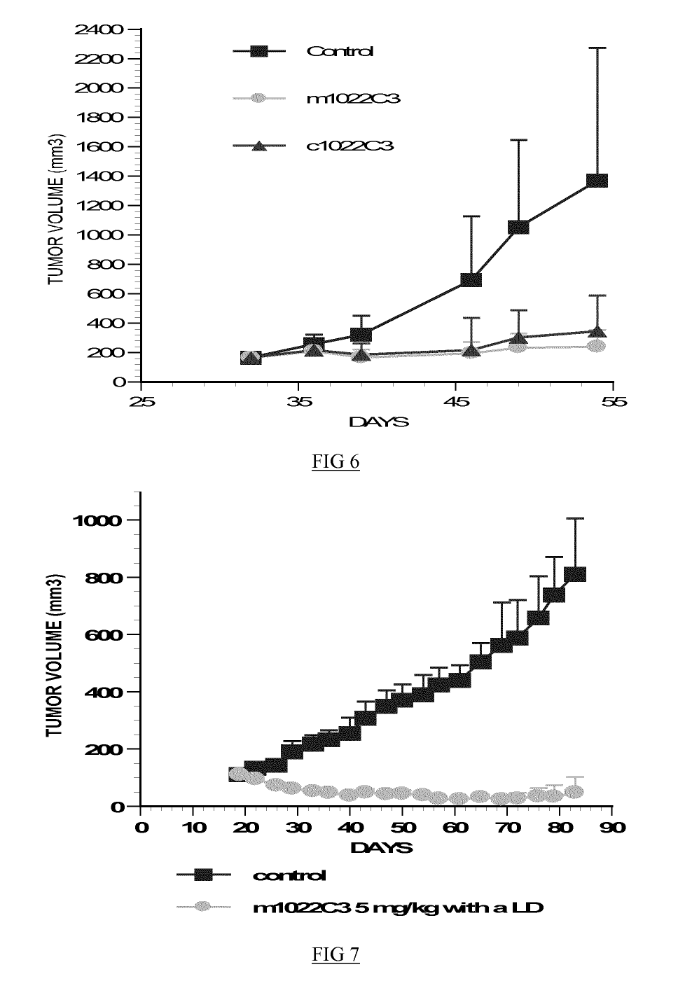

In order to compare the m1022C3 with its chimeric form, the A431 xenograft model was set up by cell engraftments on athymic mice as described above. Results presented in FIG. 6 demonstrated that the two compounds are comparable with tumour inhibitions reaching respectively 82% and 75% for m1022C3 and c1022C3.

### 7.4 CaOV3 Xenograft Model: Established Tumours

CaOV-3, an ovarian carcinoma cell line, expressing ADAM17 (ABC=20 000), was selected for in vivo evaluations. Mice were injected subcutaneously at D0 with 7×10⁶ cells. When tumours reached approximately 120 mm³ (18 days post tumour cell injection), animals were divided into two groups of 5 mice with comparable tumour size and treated intraperitoneally with a loading dose of 10 mg/kg and then weekly with maintenance doses of 5 mg/kg of m1022C3 monoclonal antibody. A control group received only the vehicle as previous experiments performed in this model demonstrated that no difference in tumour growth was observed between mice treated with vehicle and mice injected with an isotype control. The mice were followed for the observation of xenograft growth rate. Tumour volume was calculated by the formula: π/6×length×width×height.

The results obtained were summarized in FIG. 7. They showed a dramatic tumour inhibition mediated by the m1022C3 mAb with tumour regressions observed in all treated mice and complete regressions achieved in 1 out of 5 mice since D69. The tumour inhibition reached 94% at D84.

### 7.5 OVISE Xenograft Model: Established Tumours

OVISE, an ovarian carcinoma cell line, expressing ADAM17 (ABC=49 000), was selected for in vivo evaluations. Mice were injected subcutaneously at D0 with 7×10⁶ cells. When tumours reached approximately 100 mm³ (28 days post tumour cell injection), animals were divided into two groups of 6 mice with comparable tumour size and treated intraperitoneally with a loading dose of 10 mg/kg and then weekly with maintenance doses of 5 mg/kg of m1022C3 monoclonal antibody. A control group received only the vehicle as previous experiments performed in this model demonstrated that no difference in tumour growth was observed between mice treated with vehicle and mice injected with an isotype control. The mice were followed for the observation of xenograft growth rate. Tumour volume was calculated by the formula: π/6×length×width×height.

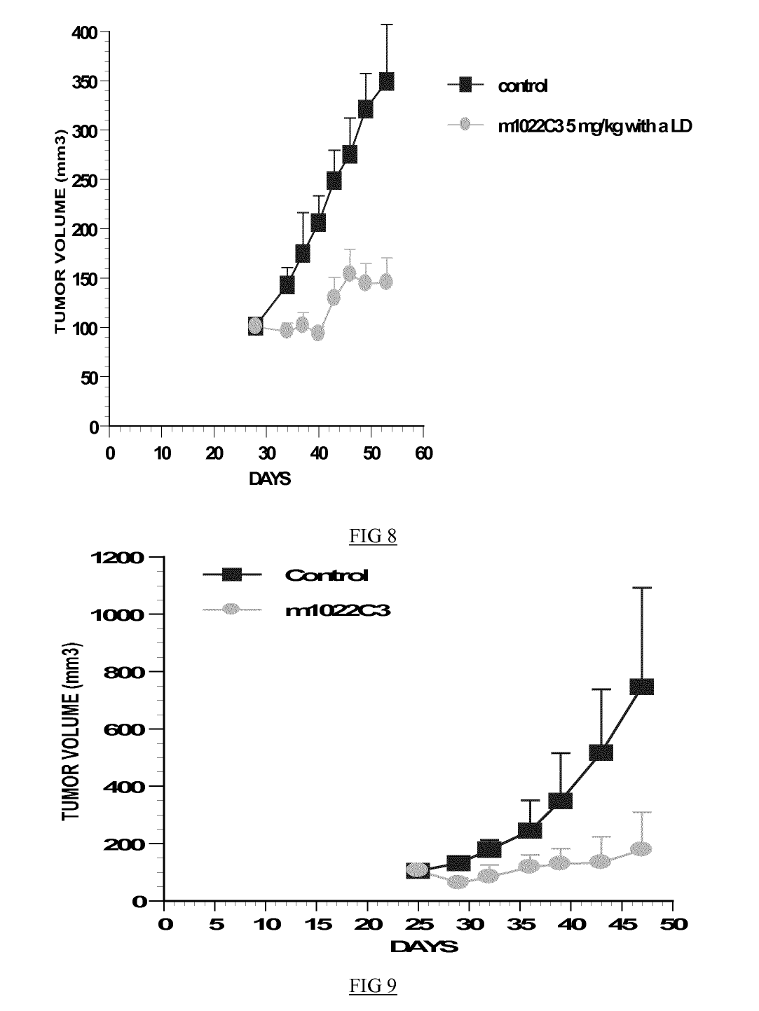

The results obtained were summarized in FIG. 8. They showed a dramatic tumour inhibition mediated by the mAb m1022C3 with tumour regressions observed in all treated mice with a tumour inhibition reaching 58% at D53.

### 7.6 BxPC3 Xenograft Tumours

BxPC3, a pancreatic carcinoma cell line, expressing ADAM17 (ABC=12 600), was selected for in vivo evaluations. Mice were injected subcutaneously at D0 with 7×10⁶ cells. When tumours reached approximately 100 mm³ (25 days post tumour cell injection), animals were divided into two groups of 6 mice with comparable tumour size and treated intraperitoneally with a loading dose of 20 mg/kg and then weekly with maintenance doses of 10 mg/kg of m1022C3 monoclonal antibody. A control group received only the vehicle as previous experiments performed in this model demonstrated that no difference in tumour growth was observed between mice treated with vehicle and mice injected with an isotype control. The mice were followed for the observation of xenograft growth rate. Tumour volume was calculated by the formula: π/6×length×width×height.

The results obtained were summarized in FIG. 9. They showed a dramatic tumour inhibition, reaching 76% at D47 mediated by the mAb m1022C3.

### 7.7 Comparison of the Murine 1022C3 (m1022C3) with its Humanized Form (hz1022C3) on the CaOV3 Xenograft Model

In order to compare the m1022C3 with its humanized form, the CaOV3 xenograft model was set up by cell engraftments on SCID mice as described above.

CaOV-3, an ovarian carcinoma cell line, expressing ADAM17 (ABC=20 000), was selected for in vivo evaluations. Mice were injected subcutaneously at D0 with 7×10⁶ cells. When tumours reached approximately 120 mm³ (198 days post tumour cell injection), animals were divided into two groups of 5 mice with comparable tumour size and treated intraperitoneally with a loading dose of 10 mg/kg and then weekly with maintenance doses of 5 mg/kg of m1022C3 and hz1022C3 monoclonal antibody or 2.5 mg/kg and then weekly with maintenance doses of 1.25 mg/kg. A control group received only the vehicle as previous experiments performed in this model demonstrated that no difference in tumour growth was observed between mice treated with vehicle and mice injected with an isotype control. The mice were followed for the observation of xenograft growth rate. Tumour volume was calculated by the formula: π/6×length×width×height.

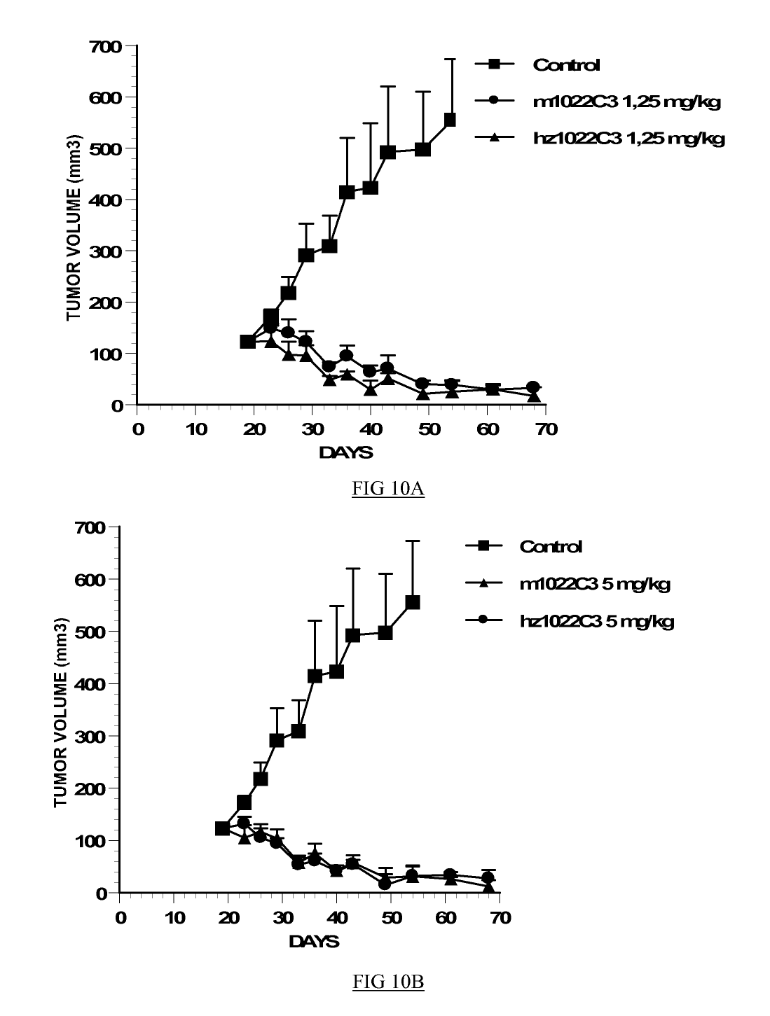

Results presented in FIGS. 10A and 10B demonstrated that the two compounds are comparable with tumour inhibitions reaching respectively 93% and 94% for m1022C3 and hz1022C3 when used at 1.25 mg/kg and 94% for both antibodies when used at 5 mg/kg.

1. A humanized antibody that binds to an ADAM 17 epitope, or an antigen binding fragment thereof, said antibody comprising:
a) a heavy chain variable domain having:
i) complementary determining regions CDR-H1, CDR-H2 and CDR-H3 of sequences SEQ ID Nos. 1, 2 and 3, respectively, and
ii) framework regions FR1, FR2 and FR3 derived from the human germline IGHV4-4*07 (SEQ ID No. 18), and
iii) framework region FR4 derived from the human germline IGHJ6*01 (SEQ ID No. 20); and
b) a light chain variable domain having:
i) complementary determining regions CDR-L1, CDR-L2 and CDR-L3 of sequences SEQ ID Nos. 4, 5 and 6, respectively, and
ii) framework regions FR1, FR2 and FR3 derived from the human germline IGKV1-39*01 (SEQ ID No. 19), and
iii) framework region FR4 derived from the human germline IGKJ4*01 (SEQ ID No. 21).

2. The humanized antibody or antigen binding fragment thereof of claim 1, wherein i) the heavy chain variable domain consists of the sequence SEQ ID No. 7 and ii) the light chain variable domain consists of the sequence SEQ ID No. 8.

3. The humanized antibody or antigen binding fragment thereof of claim 1, wherein the heavy chain variable domain consists of the sequence SEQ ID No. 9, or any sequence exhibiting at least 80% identity with SEQ ID No. 9.

4. The humanized antibody or antigen binding fragment thereof of claim 1, wherein the light chain variable domain consists of the sequence SEQ ID No. 10, or any sequence exhibiting at least 80% identity with SEQ ID No. 10.

5. The humanized antibody or antigen binding fragment thereof of claim 1, wherein it comprises a heavy chain variable domain of sequence SEQ ID No. 9 and a light chain variable domain of sequence SEQ ID No. 10.

6. The humanized antibody or antigen binding fragment thereof of claim 1 wherein said antigen binding fragment is a F(ab), a F(ab′), a F(ab′)₂ or a scFv fragment.

7. The humanized antibody or an antigen binding fragment thereof of claim 1 wherein it is of the IgG1, IgG2, IgG3, or IgG4 isotype.

8. The humanized antibody or antigen binding fragment thereof of claim 7, said humanized antibody comprising:
a) a heavy chain of sequence selected in the group consisting of sequences SEQ ID No. 11, 12, 13, and 14 and
b) a light chain of sequence SEQ ID No. 15.

9. A humanized antibody, or an antigen binding fragment thereof, wherein said humanized antibody is an affinity matured mutant of the humanized antibody of claim 1.

10. (canceled)

11. A pharmaceutical composition comprising as an active ingredient the humanized antibody or antigen binding fragment thereof of claim 1.

12.-14. (canceled)

15. An isolated nucleic acid coding for the humanized antibody or antigen binding fragment thereof of claim 1.

16. The isolated nucleic acid of claim 15, wherein the antibody, or antigen binding fragment thereof, comprises a heavy chain variable domain comprising the sequence SEQ ID No. 16 and a light chain variable domain comprising the sequence SEQ ID No. 17.

17. A method of treatment of cancer, said method comprising administering to a patient in need thereof the humanized antibody or antigen binding fragment thereof of claim 1.

18. The method of claim 17, wherein said cancer is a cancer selected among prostate cancer, lung cancer, breast cancer, ovarian cancer, pancreatic cancer, head and neck cancer, cancer of the kidney, and colon cancer.

19. A method of treatment of cancer, said method comprising administering to a patient in need thereof the pharmaceutical composition of claim 11.

20. The method of claim 19, wherein said cancer is a cancer selected among prostate cancer, lung cancer, breast cancer, ovarian cancer, pancreatic cancer, head and neck cancer, cancer of the kidney, and colon cancer.


SEQUENCE LISTING (57 sequences)

SEQ ID NO: 1 - protein, 10 aa, Artificial Sequence
  Description of Artificial Sequence Synthetic Humanized ADAM17 antibody CDR-H1 sequence
GFSLDTSGMG

SEQ ID NO: 2 - protein, 7 aa, Artificial Sequence
  Description of Artificial Sequence Synthetic Humanized ADAM17 antibody CDR-H2 sequence
IYWDDDK

SEQ ID NO: 3 - protein, 15 aa, Artificial Sequence
  Description of Artificial Sequence Synthetic Humanized ADAM17 antibody CDR-H3 sequence
ARRSHFGNYYYAMDY

SEQ ID NO: 4 - protein, 6 aa, Artificial Sequence
  Description of Artificial Sequence Synthetic Humanized ADAM17 antibody CDR-L1 sequence
QDISSY

SEQ ID NO: 5 - protein, 3 aa, Artificial Sequence
  Description of Artificial Sequence Synthetic Humanized ADAM17 antibody CDR-L2 sequence
YTS

SEQ ID NO: 6 - protein, 9 aa, Artificial Sequence
  Description of Artificial Sequence Synthetic Humanized ADAM17 antibody CDR-L3 sequence
QQGKTLPLT

SEQ ID NO: 7 - protein, 123 aa, Artificial Sequence
  Description of Artificial Sequence Synthetic VH consensus sequence
  MOD_RES (3)..(3): Thr or Gln
  MOD_RES (5)..(5): Lys or Gln
  MOD_RES (11)..(11): Ile or Leu
  MOD_RES (12)..(12): Leu or Val
  MOD_RES (13)..(13): Gln or Lys
  MOD_RES (16)..(16): Gln or Glu
  MOD_RES (23)..(23): Ser or Thr
  MOD_RES (24)..(24): Phe or Val
  MOD_RES (36)..(36): Val or Trp
  MOD_RES (43)..(43): Ser or Ala
  MOD_RES (50)..(50): Leu or Ile
  MOD_RES (51)..(51): Ala or Gly
  MOD_RES (52)..(52): His or Arg
  MOD_RES (60)..(60): Arg or Asn
  MOD_RES (69)..(69): Leu or Val
  MOD_RES (71)..(71): Ile or Met
  MOD_RES (73)..(73): Lys or Val
  MOD_RES (77)..(77): Ser or Lys
  MOD_RES (80)..(80): Val or Phe
  MOD_RES (81)..(81): Phe or Ser
  MOD_RES (84)..(84): Ile or Leu
  MOD_RES (85)..(85): Thr or Ser
  MOD_RES (87)..(87): Leu or Val
  MOD_RES (88)..(88): Asp or Thr
  MOD_RES (89)..(89): Thr or Ala
  MOD_RES (94)..(94): Thr or Val
  MOD_RES (118)..(118): Ser or Thr
  MOD_RES (122)..(122): Thr or Ser
QVXLXESGPGXXXPSXTLSLTCXXSGFSLDTSGMGXSWIRQPXGKGLEWXXXIYWDDDKXYNPSLKSRXTXSXDTSXNQXXLKXXSXXXADTAXYYCARRSHFGNYYYAMDYWGQGTXVTVXS

SEQ ID NO: 8 - protein, 107 aa, Artificial Sequence
  Description of Artificial Sequence Synthetic VL consensus sequence
  MOD_RES (2)..(2): Val or Ile
  MOD_RES (7)..(7): Thr or Ser
  MOD_RES (8)..(8): Thr or Pro
  MOD_RES (15)..(15): Leu or Val
  MOD_RES (22)..(22): Ser or Thr
  MOD_RES (41)..(41): Asp or Gly
  MOD_RES (42)..(42): Gly or Lys
  MOD_RES (43)..(43): Thr or Ala
  MOD_RES (44)..(44): Phe or Pro
  MOD_RES (49)..(49): Phe or Tyr
  MOD_RES (53)..(53): Arg or Ser
  MOD_RES (61)..(61): Lys or Arg
  MOD_RES (62)..(62): Tyr or Phe
  MOD_RES (71)..(71): Tyr or Phe
  MOD_RES (72)..(72): Ser or Thr
  MOD_RES (77)..(77): Asn or Ser
  MOD_RES (79)..(79): Glu or Gln
  MOD_RES (80)..(80): Gln or Pro
  MOD_RES (83)..(83): Ile or Phe
  MOD_RES (85)..(85): Ala or Thr
  MOD_RES (87)..(87): Phe or Tyr
  MOD_RES (100)..(100): Ala or Gly
  MOD_RES (104)..(104): Leu or Val
  MOD_RES (106)..(106): Leu or Ile
DXQMTQXXSSLSASXGDRVTIXCRASQDISSYLNWYQQKPXXXXKLLIXYTSXLQSGVPSXXSGSGSGTDXXLTISXLXXEDXAXYXCQQGKTLPLTFGXGTKXEXK

SEQ ID NO: 9 - protein, 123 aa, Artificial Sequence
  Description of Artificial Sequence Synthetic Humanized ADAM17 antibody VH variable sequence
QVQLQESGPGLVKPSETLSLTCTVSGFSLDTSGMGVSWIRQPAGKGLEWIGHIYWDDDKRYNPSLKSRVTMSKDTSSNQVSLKLSSVTAADTAVYYCARRSHFGNYYYAMDYWGQGTTVTVSS

SEQ ID NO: 10 - protein, 107 aa, Artificial Sequence
  Description of Artificial Sequence Synthetic Humanized ADAM17 antibody VL variable sequence
DIQMTQSPSSLSASVGDRVTITCRASQDISSYLNWYQQKPGKAPKLLIFYTSRLQSGVPSRFSGSGSGTDFTLTISSLQPEDFATYYCQQGKTLPLTFGGGTKVEIK

SEQ ID NO: 11 - protein, 452 aa, Artificial Sequence
  Description of Artificial Sequence Synthetic Humanized ADAM17 antibody VH full IgG1 sequence
QVQLQESGPGLVKPSETLSLTCTVSGFSLDTSGMGVSWIRQPAGKGLEWIGHIYWDDDKRYNPSLKSRVTMSKDTSSNQVSLKLSSVTAADTAVYYCARRSHFGNYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 448 aa, Artificial Sequence
  Description of Artificial Sequence Synthetic Humanized ADAM17 antibody VH full IgG2 sequence
QVQLQESGPGLVKPSETLSLTCTVSGFSLDTSGMGVSWIRQPAGKGLEWIGHIYWDDDKRYNPSLKSRVTMSKDTSSNQVSLKLSSVTAADTAVYYCARRSHFGNYYYAMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 13 - protein, 469 aa, Artificial Sequence
  Description of Artificial Sequence Synthetic Humanized ADAM17 antibody VH full IgG3 sequence
QVQLQESGPGLVKPSETLSLTCTVSGFSLDTSGMGVSWIRQPAGKGLEWIGHIYWDDDKRYNPSLKSRVTMSKDTSSNQVSLKLSSVTAADTAVYYCARRSHFGNYYYAMDYWGQGTTVTVSSASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 14 - protein, 449 aa, Artificial Sequence
  Description of Artificial Sequence Synthetic Humanized ADAM17 antibody VH full IgG4 sequence
QVQLQESGPGLVKPSETLSLTCTVSGFSLDTSGMGVSWIRQPAGKGLEWIGHIYWDDDKRYNPSLKSRVTMSKDTSSNQVSLKLSSVTAADTAVYYCARRSHFGNYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 15 - protein, 214 aa, Artificial Sequence
  Description of Artificial Sequence Synthetic Humanized ADAM17 antibody VL full sequence
DIQMTQSPSSLSASVGDRVTITCRASQDISSYLNWYQQKPGKAPKLLIFYTSRLQSGVPSRFSGSGSGTDFTLTISSLQPEDFATYYCQQGKTLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - DNA, 369 bp, Artificial Sequence
  Description of Artificial Sequence Synthetic Humanized ADAM17 antibody VH variable sequence
caagttcagttgcaggaatccggtcctggtcttgttaagccttccgagactttgtccctcacttgtaccgtgtccggctttagccttgacactagcggaatgggagtgagttggatcaggcagcccgcaggaaaaggcctggagtggattggccatatatactgggatgatgataaaaggtacaaccccagcctgaagtcacgtgtgacaatgagcaaagacacatctagcaatcaggtgagtctgaaactgtcttctgtgaccgccgctgacaccgctgtgtactactgcgcccggcgctctcattttgggaattattattatgccatggactattgggggcagggcacaaccgtcaccgtgtcatca

SEQ ID NO: 17 - DNA, 321 bp, Artificial Sequence
  Description of Artificial Sequence Synthetic Humanized ADAM17 antibody VL variable sequence
gacatccagatgacccaatccccatcatccttgagcgcttcagtgggagatagggtaaccatcacctgcagggcatctcaggatatcagctcttacctgaactggtaccagcagaagcccggaaaggcccctaaattgctgattttctacaccagccgcctgcagagtggagttcctagtcggttttccggcagtggctctgggactgatttcactctgactattagctctctgcagcccgaagacttcgccacatattattgccagcaggggaagacactccccctgacatttggcgggggcacaaaagtggagataaag

SEQ ID NO: 18 - protein, 97 aa, Artificial Sequence
  Description of Artificial Sequence Synthetic Framework region derived from the human germline IGHV4-4*07 sequence
QVQLQESGPGLVKPSETLSLTCTVSGGSISSYYWSWIRQPAGKGLEWIGRIYTSGSTNYNPSLKSRVTMSVDTSKNQFSLKLSSVTAADTAVYYCAR

SEQ ID NO: 19 - protein, 95 aa, Artificial Sequence
  Description of Artificial Sequence Synthetic Framework region derived from the human germline IGKV1-39*01 sequence
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 20 - protein, 20 aa, Artificial Sequence
  Description of Artificial Sequence Synthetic Framework region FR4 derived from the human germline IGHJ6*01 sequence
YYYYYGMDVWGQGTTVTVSS

SEQ ID NO: 21 - protein, 12 aa, Artificial Sequence
  Description of Artificial Sequence Synthetic Framework region FR4 derived from the human germline IGKJ4*01 sequence
LTFGGGTKVEIK

SEQ ID NO: 22 - protein, 824 aa, Homo sapiens
  ADAM17
MRQSLLFLTSVVPFVLAPRPPDDPGFGPHQRLEKLDSLLSDYDILSLSNIQQHSVRKRDLQTSTHVETLLTFSALKRHFKLYLTSSTERFSQNFKVVVVDGKNESEYTVKWQDFFTGHVVGEPDSRVLAHIRDDDVIIRINTDGAEYNIEPLWRFVNDTKDKRMLVYKSEDIKNVSRLQSPKVCGYLKVDNEELLPKGLVDREPPEELVHRVKRRADPDPMKNTCKLLVVADHRFYRYMGRGEESTTTNYLIELIDRVDDIYRNTSWDNAGFKGYGIQIEQIRILKSPQEVKPGEKHYNMAKSYPNEEKDAWDVKMLLEQFSFDIAEEASKVCLAHLFTYQDFDMGTLGLAYVGSPRANSHGGVCPKAYYSPVGKKNIYLNSGLTSTKNYGKTILTKEADLVTTHELGHNFGAEHDPDGLAECAPNEDQGGKYVMYPIAVSGDHENNKMFSNCSKQSIYKTIESKAQECFQERSNKVCGNSRVDEGEECDPGIMYLNNDTCCNSDCTLKEGVQCSDRNSPCCKNCQFETAQKKCQEAINATCKGVSYCTGNSSECPPPGNAEDDTVCLDLGKCKDGKCIPFCEREQQLESCACNETDNSCKVCCRDLSGRCVPYVDAEQKNLFLRKGKPCTVGFCDMNGKCEKRVQDVIERFWDFIDQLSINTFGKFLADNIVGSVLVFSLIFWIPFSILVHCVDKKLDKQYESLSLFHPSNVEMLSSMDSASVRIIKPFPAPQTPGRLQPAPVIPSAPAAPKLDHQRMDTIQEDPSTDSHMDEDGFEKDPFPNSSTAAKSFEDLTDHPVTRSEKAASFKLQRQNRVDSKETEC

SEQ ID NO: 23 - protein, 260 aa, Artificial Sequence
  Description of Artificial Sequence Synthetic ADAM17 MP domain sequence
RADPDPMKNTCKLLVVADHRFYRYMGRGEESTTTNYLIELIDRVDDIYRNTSWDNAGFKGYGIQIEQIRILKSPQEVKPGEKHYNMAKSYPNEEKDAWDVKMLLEQFSFDIAEEASKVCLAHLFTYQDFDMGTLGLAYVGSPRANSHGGVCPKAYYSPVGKKNIYLNSGLTSTKNYGKTILTKEADLVTTHELGHNFGAEHDPDGLAECAPNEDQGGKYVMYPIAVSGDHENNKMFSNCSKQSIYKTIESKAQECFQERS

SEQ ID NO: 24 - protein, 89 aa, Artificial Sequence
  Description of Artificial Sequence Synthetic ADAM17 DI domain sequence
NKVCGNSRVDEGEECDPGIMYLNNDTCCNSDCTLKEGVQCSDRNSPCCKNCQFETAQKKCQEAINATCKGVSYCTGNSSECPPPGNAED

SEQ ID NO: 25 - protein, 108 aa, Artificial Sequence
  Description of Artificial Sequence Synthetic ADAM17 MPD domain sequence
DTVCLDLGKCKDGKCIPFCEREQQLESCACNETDNSCKVCCRDLSGRCVPYVDAEQKNLFLRKGKPCTVGFCDMNGKCEKRVQDVIERFWDFIDQLSINTFGKFLADN

SEQ ID NO: 26 - protein, 15 aa, Artificial Sequence
  Description of Artificial Sequence Synthetic Peptide_001 sequence
DTVCLDLGKCKDGKC

SEQ ID NO: 27 - protein, 15 aa, Artificial Sequence
  Description of Artificial Sequence Synthetic Peptide_002 sequence
CLDLGKCKDGKCIPF

SEQ ID NO: 28 - protein, 15 aa, Artificial Sequence
  Description of Artificial Sequence Synthetic Peptide_003 sequence
LGKCKDGKCIPFCER

SEQ ID NO: 29 - protein, 15 aa, Artificial Sequence
  Description of Artificial Sequence Synthetic Peptide_004 sequence
CKDGKCIPFCEREQQ

SEQ ID NO: 30 - protein, 15 aa, Artificial Sequence
  Description of Artificial Sequence Synthetic Peptide_005 sequence
GKCIPFCEREQQLES

SEQ ID NO: 31 - protein, 15 aa, Artificial Sequence
  Description of Artificial Sequence Synthetic Peptide_006 sequence
IPFCEREQQLESCAC

SEQ ID NO: 32 - protein, 15 aa, Artificial Sequence
  Description of Artificial Sequence Synthetic Peptide_007 sequence
CEREQQLESCACNET

SEQ ID NO: 33 - protein, 15 aa, Artificial Sequence
  Description of Artificial Sequence Synthetic Peptide_008 sequence
EQQLESCACNETDNS

SEQ ID NO: 34 - protein, 15 aa, Artificial Sequence
  Description of Artificial Sequence Synthetic Peptide_009 sequence
LESCACNETDNSCKV

SEQ ID NO: 35 - protein, 15 aa, Artificial Sequence
  Description of Artificial Sequence Synthetic Peptide_010 sequence
CACNETDNSCKVCCR

SEQ ID NO: 36 - protein, 15 aa, Artificial Sequence
  Description of Artificial Sequence Synthetic Peptide_011 sequence
NETDNSCKVCCRDLS

SEQ ID NO: 37 - protein, 15 aa, Artificial Sequence
  Description of Artificial Sequence Synthetic Peptide_012 sequence
DNSCKVCCRDLSGRC

SEQ ID NO: 38 - protein, 15 aa, Artificial Sequence
  Description of Artificial Sequence Synthetic Peptide_013 sequence
CKVCCRDLSGRCVPY

SEQ ID NO: 39 - protein, 15 aa, Artificial Sequence
  Description of Artificial Sequence Synthetic Peptide_014 sequence
CCRDLSGRCVPYVDA

SEQ ID NO: 40 - protein, 15 aa, Artificial Sequence
  Description of Artificial Sequence Synthetic Peptide_015 sequence
DLSGRCVPYVDAEQK

SEQ ID NO: 41 - protein, 15 aa, Artificial Sequence
  Description of Artificial Sequence Synthetic Peptide_016 sequence
GRCVPYVDAEQKNLF

SEQ ID NO: 42 - protein, 15 aa, Artificial Sequence
  Description of Artificial Sequence Synthetic Peptide_017 sequence
VPYVDAEQKNLFLRK

SEQ ID NO: 43 - protein, 15 aa, Artificial Sequence
  Description of Artificial Sequence Synthetic Peptide_018 sequence
VDAEQKNLFLRKGKP

SEQ ID NO: 44 - protein, 15 aa, Artificial Sequence
  Description of Artificial Sequence Synthetic Peptide_019 sequence
EQKNLFLRKGKPCTV

SEQ ID NO: 45 - protein, 15 aa, Artificial Sequence
  Description of Artificial Sequence Synthetic Peptide_020 sequence
NLFLRKGKPCTVGFC

SEQ ID NO: 46 - protein, 15 aa, Artificial Sequence
  Description of Artificial Sequence Synthetic Peptide_021 sequence
LRKGKPCTVGFCDMN

SEQ ID NO: 47 - protein, 15 aa, Artificial Sequence
  Description of Artificial Sequence Synthetic Peptide_022 sequence
GKPCTVGFCDMNGKC

SEQ ID NO: 48 - protein, 15 aa, Artificial Sequence
  Description of Artificial Sequence Synthetic Peptide_023 sequence
CTVGFCDMNGKCEKR

SEQ ID NO: 49 - protein, 15 aa, Artificial Sequence
  Description of Artificial Sequence Synthetic Peptide_024 sequence
GFCDMNGKCEKRVQD

SEQ ID NO: 50 - protein, 15 aa, Artificial Sequence
  Description of Artificial Sequence Synthetic Peptide_025 sequence
DMNGKCEKRVQDVIE

SEQ ID NO: 51 - protein, 15 aa, Artificial Sequence
  Description of Artificial Sequence Synthetic Peptide_026 sequence
GKCEKRVQDVIERFW

SEQ ID NO: 52 - protein, 15 aa, Artificial Sequence
  Description of Artificial Sequence Synthetic Peptide_027 sequence
EKRVQDVIERFWDFI

SEQ ID NO: 53 - protein, 15 aa, Artificial Sequence
  Description of Artificial Sequence Synthetic Peptide_028 sequence
VQDVIERFWDFIDQL

SEQ ID NO: 54 - protein, 15 aa, Artificial Sequence
  Description of Artificial Sequence Synthetic Peptide_029 sequence
VIERFWDFIDQLSIN

SEQ ID NO: 55 - protein, 15 aa, Artificial Sequence
  Description of Artificial Sequence Synthetic Peptide_030 sequence
RFWDFIDQLSINTFG

SEQ ID NO: 56 - protein, 15 aa, Artificial Sequence
  Description of Artificial Sequence Synthetic Peptide_031 sequence
DFIDQLSINTFGKFL

SEQ ID NO: 57 - protein, 15 aa, Artificial Sequence
  Description of Artificial Sequence Synthetic Peptide_032 sequence
IDQLSINTFGKFLAD